(19) United States
(12) Patent Application Publication
(10) Pub. No.: US 2023/0340018 A1
(43) Pub. Date: Oct. 26, 2023

(54) MPRO CYSTEINE PROTEASE INHIBITORS

(71) Applicant: ANIXA BIOSCIENCES, INC., San Jose, CA (US)

(72) Inventors: Wolfgang RICHTER, München (DE); Muhammad ABBAS, Lahore (PK); Lutz WEBER, Stuttgart (DE); Amit KUMAR, San Jose, CA (US)

(21) Appl. No.: 18/041,836

(22) PCT Filed: Aug. 17, 2021
(86) PCT No.: PCT/US2021/046311

§ 371 Date: Feb. 16, 2023

Related U.S. Application Data
(60) Provisional application No. 63/067,237, filed on Aug. 18, 2020.

(51) Int. Cl. C07K 5/097; A61P 31/14

(52) U.S. Cl. CPC C07K 5/0821; A61P 31/14; A61K 38/00

(57) ABSTRACT

Described herein are Mpro cysteine protease inhibitors and methods of utilizing such inhibitors in the treatment of diseases, disorders, or conditions. Also described herein are pharmaceutical compositions containing such compounds.

### CROSS-REFERENCE

This application claims benefit of U.S. Provisional Pat. Application No. 63/067,237, filed on Aug. 18, 2020 which is incorporated herein by reference in its entirety.

### BACKGROUND OF THE INVENTION

Coronaviruses infect humans and other animals and cause a variety of highly prevalent and severe diseases, including severe acute respiratory syndrome (SARS), Middle East respiratory syndrome (MERS), and coronavirus disease 2019 (COVID-19). Main protease (M^{pro}) of SARS-CoV-2: M^{pro} is a key enzyme of coronaviruses and has a pivotal role in mediating viral replication and transcription. Small molecule inhibitors of SARS-CoV-2 provide an attractive drug target to these coronavirus infections.

### SUMMARY OF THE INVENTION

In one aspect, provided herein are compounds of Formula (I′):

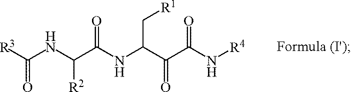

wherein:
R¹ is selected from

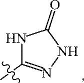

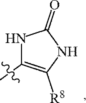

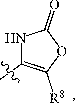

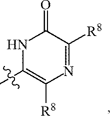

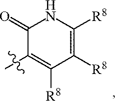

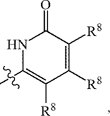

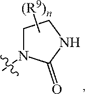

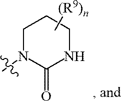

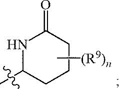

R² is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉ heteroaryl, and -C₁₋₆alkyl-C₁₋₉heteroaryl, wherein C₁₋₆alkyl, C₂₋₆alkenyl, C₂₋₆alkynyl, C₃₋₆ cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)_{2N}(R⁵)(R⁶)—;
R⁴ is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉ heteroaryl, and -C₁₋₆alkyl-C₁₋₉heteroaryl, wherein C₁₋₆alkyl, C₂₋₆alkenyl, C₂₋₆alkynyl, C₃₋₆ cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆ haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
each R⁵ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally sub stituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁶ is independently selected from hydrogen, C₁₋₆alkyl, and C₁₋₆haloalkyl; and
each R⁷ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁸ is independently selected from hydrogen, halogen, C₁₋₆alkyl, C₁₋₆haloalkyl, —N(R⁵)(R⁶), —C(O)R⁷, —C(O)OR⁵, —C(O)N(R⁵)(R⁶), —S(O)₂R⁷, and —S(O)_{2N}(R⁵)(R⁶)—; and
each R⁹ is independently selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)_{2N}(R⁵)(R⁶)—; and
n is 0, 1, 2, 3, or 4.;
or a pharmaceutically acceptable salt or solvate thereof.

In another aspect, provided herein are compounds of Formula (I):

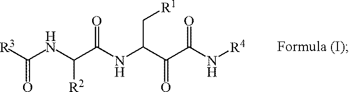

wherein:
R¹ is selected from

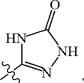

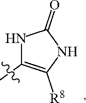

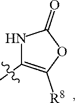

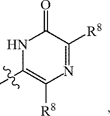

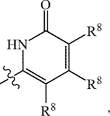

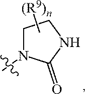

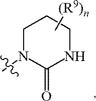

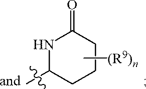

R² is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)_{2N}(R⁵)(R⁶)—;
R⁴ is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
each R⁵ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally sub stituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁶ is independently selected from hydrogen, C₁₋₆alkyl, and C₁₋₆haloalkyl; and
each R⁷ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁸ is independently selected from hydrogen, halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)_{2N}(R⁵)(R⁶)—; and
each R⁹ is independently selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)_{2N}(R⁵)(R⁶)—; and
n is 0, 1, 2, 3, or 4.;
or a pharmaceutically acceptable salt or solvate thereof.

In some embodiments is a compound of Formula (I) or (I′), or a pharmaceutically acceptable salt or solvate thereof, whereinR⁴ is selected from C₁₋₆alkyl, C₃₋₆cycloalkyl, -C₁₋₆alkyl-C₃₋₆cycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₃₋₆cycloalkyl, -C₁₋₆alkyl-C₃₋₆cycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R⁴ is selected from C₃₋₆cycloalkyl, -C₁₋₆alkyl-C₃₋₆cycloalkyl, C₆₋₁₀aryl, and -C₁₋₆alkyl-C₆₋₁₀aryl, wherein C₃₋₆cycloalkyl, -C₁₋₆alkyl-C₃₋₆cycloalkyl, C₆₋₁₀aryl, and -C₁₋₆alkyl-C₆₋₁₀aryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R⁴ is C₃_₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R⁴ is unsubstituted C₃₋₆cycloalkyl. In some embodiments is a compound of Formula (I) or (I′), or a pharmaceutically acceptable salt or solvate thereof, wherein R⁴ is -C₁₋₆alkyl-C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R⁴ is unsubstituted -C₁₋₆alkyl-C₃₋₆cycloalkyl. In some embodiments is a compound of Formula (I) or (I′), or a pharmaceutically acceptable salt or solvate thereof, wherein R⁴ is phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R⁴ is unsubstituted phenyl. In some embodiments is a compound of Formula (I) or (I′), or a pharmaceutically acceptable salt or solvate thereof, wherein R⁴ is -C₁₋₆alkyl-phenyl optionally substituted with one, two, three, or four groups selected from halogen, C ₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R⁴ is unsubstituted -C₁₋₆alkyl-phenyl. In some embodiments is a compound of Formula (I) or (I′), or a pharmaceutically acceptable salt or solvate thereof, wherein R⁴ is

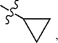

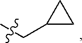

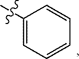

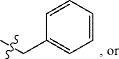

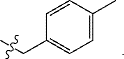

In some embodiments is a compound of Formula (I) or (I′), or a pharmaceutically acceptable salt or solvate thereof, wherein R³ is C₁₋₉heteroaryl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R³ is unsubstituted C₁₋₉heteroaryl. In some embodiments is a compound of Formula (I) or (I′), or a pharmaceutically acceptable salt or solvate thereof, wherein R³ is

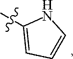

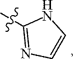

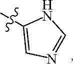

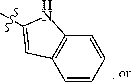

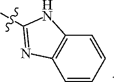

. In some embodiments is a compound of Formula (I) or (I′), or a pharmaceutically acceptable salt or solvate thereof, wherein R² is selected from C₁₋₆alkyl, -C₁₋₆alkyl-C₃₋₆cycloalkyl, -C₁₋₆alkyl-C₆₋₁₀aryl, and -C₁₋₆alkyl-C₁₋₉heteroaryl, wherein C₁₋₆alkyl, -C₁₋₆alkyl-C₃₋₆cycloalkyl, -C₁₋₆alkyl-C₆₋₁₀aryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is C₁₋₆alkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted C₁₋₆alkyl. In some embodiments is a compound of Formula (I) or (I′), or a pharmaceutically acceptable salt or solvate thereof, wherein R² is -C₁₋₆alkyl-C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted -C₁₋₆alkyl-C₃₋₆cycloalkyl. In some embodiments is a compound of Formula (I) or (I′), or a pharmaceutically acceptable salt or solvate thereof, wherein R² is -C₁₋₆alkyl-phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted -C₁₋₆alkyl-phenyl. In some embodiments is a compound of Formula (I) or (I′), or a pharmaceutically acceptable salt or solvate thereof, wherein R² is -C₁₋₆alkyl-C₁₋₉heteroaryl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted -C₁₋₆alkyl-C₁₋₉heteroaryl. In some embodiments is a compound of Formula (I) or (I′), or a pharmaceutically acceptable salt or solvate thereof, wherein R² is —CH₃, —CH₂CH₃, —C(H)(CH₃)₂, —CH₂C(H)(CH₃)₂,

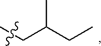

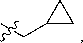

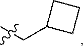

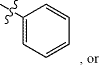

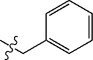

. In some embodiments is a compound of Formula (I) or (I′), or a pharmaceutically acceptable salt or solvate thereof, wherein R¹ is

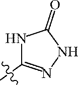

. In some embodiments is a compound of Formula (I) or (I′), or a pharmaceutically acceptable salt or solvate thereof, wherein R¹ is

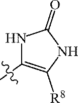

. In some embodiments is a compound of Formula (I) or (I′), or apharmaceutically acceptable salt or solvate thereof, wherein R¹ is

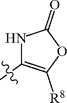

. In some embodiments is a compound of Formula (I) or (I′), or a pharmaceutically acceptable salt or solvate thereof, wherein R¹ is

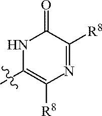

. In some embodiments is a compound of Formula (I′), or a pharmaceutically acceptable salt or solvate thereof, wherein R¹ is

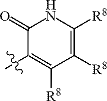

. In some embodiments is a compound of Formula (I) or (I′), or a pharmaceutically acceptable salt or solvate thereof, wherein R¹ is

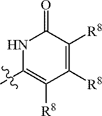

. In some embodiments is a compound of Formula (I) or (I′), or a pharmaceutically acceptable salt or solvate thereof, wherein each R⁸ is hydrogen. In some embodiments is a compound of Formula (I) or (I′), or a pharmaceutically acceptable salt or solvate thereof, wherein R¹ is

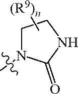

. In some embodiments is a compound of Formula (I) or (I′), or a pharmaceutically acceptable salt or solvate thereof, wherein R¹ is

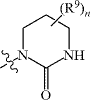

. In some embodiments is a compound of Formula (I) or (I′), or a pharmaceutically acceptable salt or solvate thereof, wherein R¹ is

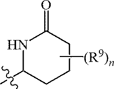

. In some embodiments is a compound of Formula (I) or (I′), or a pharmaceutically acceptable salt or solvate thereof, wherein n is 0.

In another aspect described herein is a pharmaceutical composition comprising a compound of Formula (I) or (I′), or a pharmaceutically acceptable salt or solvate thereof, and a pharmaceutically acceptable excipient.

In some embodiments described herein is a method of treating a coronavirus infection in an individual in need thereof, comprising administering to the individual a therapeutically effective amount of a compound of Formula (I) or (I′), or a pharmaceutically acceptable salt or solvate thereof. In some embodiments described herein is a method of treating a coronavirus infection in an individual in need thereof, comprising administering to the individual a therapeutically effective amount of a compound of Formula (I) or (I′), or a pharmaceutically acceptable salt or solvate thereof, wherein the coronavirus infection is severe acute respiratory syndrome coronavirus 2 (SARS-CoV-2). In some embodiments described herein is a method of treating a coronavirus infection in an individual in need thereof, comprising administering to the individual a therapeutically effective amount of a compound of Formula (I) or (I′), or a pharmaceutically acceptable salt or solvate thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the context of this disclosure, a number of terms shall be utilized.

Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4^{th} Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer’s specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to a straight or branched hydrocarbon chain radical consisting solely of carbon and hydrogen atoms, containing no unsaturation. In some embodiments, the “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆alkyl” or similar designations. By way of example only, “C₁-C₆alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, and hexyl. Alkyl groups can be substituted or unsubstituted. Depending on the structure, an alkyl group can be a monoradical or a diradical (i.e., an alkylene group).

An “alkoxy” refers to a “-O-alkyl” group, where alkyl is as defined herein.

The term “alkenyl” refers to a straight or branched hydrocarbon chain radical group consisting solely of carbon and hydrogen atoms, containing at least one carbon-carbon double bond. Non-limiting examples of an alkenyl group include —CH═CH₂, —C(CH₃)═CH₂, —CH═CHCH₃, —CH═C(CH₃)₂ and —C(CH₃)═CHCH₃. In some embodiments, an alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a straight or branched hydrocarbon chain radical group consisting solely of carbon and hydrogen atoms, containing at least one carbon-carbon triple bond. Non-limiting examples of an alkynyl group include —C≡CH, —C≡CCH₃, —C≡CCH₂CH₃ and —C≡CCH₂CH₂CH₃. In some embodiments, an alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

The term “alkylamine” or “alkylamino” refers to the -N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a -N(alkyl)₂ group, where alkyl is as defined herein.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group).

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

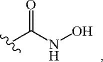

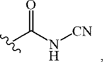

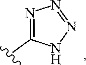

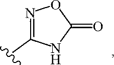

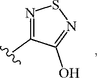

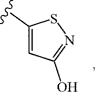

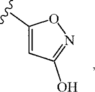

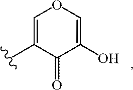

and the like.

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). In some embodiments, cycloalkyl groups include groups having from 3 to 10 ring atoms.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom.

A “heterocycloalkyl” group or “heteroalicyclic” group refers to a cycloalkyl group, wherein at least one skeletal ring atom is a heteroatom selected from nitrogen, oxygen and sulfur. The radicals may be fused with an aryl or heteroaryl. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The term “halo” or, alternatively, “halogen” means fluoro, chloro, bromo and iodo.

The term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

As used herein, the sub stituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

“Optional” or “optionally” means that a subsequently described event or circumstance may or may not occur and that the description includes instances when the event or circumstance occurs and instances in which it does not.

The term “optionally substituted” or “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂—alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(C₁-C₆alkyl)-, or -(C₂-C₆alkenyl)-; and each R^{s} is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

As used herein, the term “about” or “approximately” means within 20%, preferably within 10%, and more preferably within 5% of a given value or range.

The term a “therapeutically effective amount” as used herein refers to the amount of an Mpro cysteine protease inhibitor that, when administered to a mammal in need, is effective to at least partially ameliorate or to at least partially prevent conditions related to skin aging.

As used herein, the term “expression” includes the process by which polynucleotides are transcribed into mRNA and translated into peptides, polypeptides, or proteins.

The term “modulate” encompasses either a decrease or an increase in activity or expression depending on the target molecule.

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

The term “individual”, “patient”, or “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts, and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S.M et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Mpro Cysteine Protease Inhibitors

The virus SARS-CoV-2 is a virus from the corona virus family. Coronaviruses (CoV) are a large family of viruses that cause illness ranging from the common cold to more severe diseases. These viruses all share a cysteine proteinase “main protease” - Mpro that is a key enzyme of coronaviruses and has a pivotal role in mediating viral replication and transcription, making it an attractive drug target for SARS-CoV-2.

Mpro processes polyproteins that are translated from the viral RNA once the virus has entered human cells (Hilgenfeld et al. “Crystal structure of SARS-CoV-2 main protease provides a basis for design of improved α-ketoamide inhibitors”, Science 24 Vol. 368, Issue 6489, pp. 409-412). The crystal structure of the enzyme Mpro was identified at a resolution of 1.75 Angstroms showing an alpha-ketoamide type inhibitor in its active site.

The compounds of Formula (I) or (I′) described herein are Mpro cysteine protease inhibitors. The compounds of Formula (I) or (I′) described herein, and compositions comprising these compounds, are useful for the treatment of coronavirus infection including, but not limited to, COVID-19.

In some embodiments, provided herein is a compound of Formula (I′), or a pharmaceutically acceptable salt or solvate thereof:

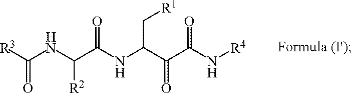

wherein:
R¹ is selected from

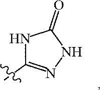

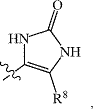

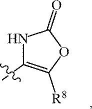

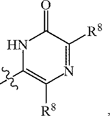

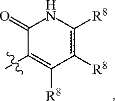

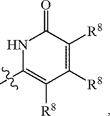

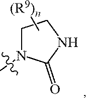

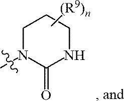

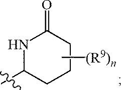

R² is selected from C₁₋₆alkyl, C₂₋₆alkenyl, C₂₋₆alkynyl, C₃₋₆cycloalkyl, -C₁₋₆alkyl-C₃₋ ₆cycloalkyl, C₂₋₉heterocycloalkyl, -C₁₋₆alkyl-C₂₋₉heterocycloalkyl, C₆₋₁₀aryl, -C₁-₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cy cloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(_{O})_{R}⁷, —C(O)OR⁵, —C(O)N(R⁵)(R⁶), —S(O)₂R⁷, and —S(O)₂N(R⁵)(R⁶)—;
R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, three, or four groups selected from halogen, C ₁-₆alkyl, C₁₋ ₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R⁴ is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁-₆alkyl, C₂₋₆alkenyl, C₂₋₆alkynyl, C₃₋₆cycloalkyl, -C₁₋₆alkyl-C₃₋₆cy cloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(_{O})_{R}⁷, —C(O)OR⁵, —C(O)N(R⁵)(R⁶), —S(O)₂R⁷, and —S(O)₂N(R⁵)(R⁶)—;
each R⁵ is independently selected from hydrogen, C₁₋₆alkyl, C₁₋₆ haloalkyl, C₂₋₆alkenyl, C₂₋ ₆alkynyl, C₃₋₆cycloalkyl, C₂₋₉heterocycloalkyl, C₆₋₁₀aryl, and C₁₋₉heteroaryl, wherein C₁₋ ₆alkyl, C₂₋₆alkenyl, C₂₋₆alkynyl, C₃₋₆cycloalkyl, C₂₋₉heterocycloalkyl, C₆₋₁₀aryl, and C₁₋ ₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁶ is independently selected from hydrogen, C₁₋₆alkyl, and C₁₋₆haloalkyl; and
each R⁷ is independently selected C₁₋₆alkyl, C₂₋₆alkenyl, C₂₋₆alkynyl, C₃₋₆cycloalkyl, C₂₋ ₉heterocycloalkyl, C₆₋₁₀aryl, and C₁₋₉heteroaryl, wherein C₁₋₆alkyl, C₂₋₆alkenyl, C₂₋ ₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁸ is independently selected from hydrogen, halogen, C₁₋₆alkyl, C₁₋₆haloalkyl, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and
each R⁹ is independently selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and n is 0, 1, 2, 3, or 4.

In some embodiments, provided herein is a compound of Formula (I), or a pharmaceutically acceptable salt or solvate thereof:

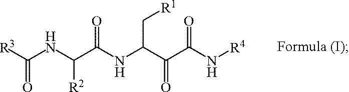

wherein:
R¹ is selected from

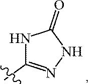

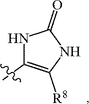

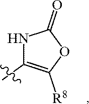

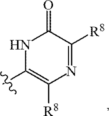

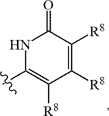

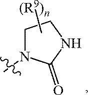

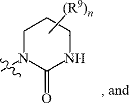

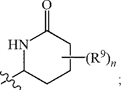

R² is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋ ₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R³ is selected from C₃-₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, three, or four groups selected from halogen, C ₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R⁴ is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁-₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
each R⁵ is independently selected from hydrogen, C₁₋₆alkyl, C₁₋₆ haloalkyl, C₂₋₆alkenyl, C₂₋ ₆alkynyl, C₃₋₆cycloalkyl, C₂₋₉heterocycloalkyl, C₆₋₁₀aryl, and C₁₋₉heteroaryl, wherein C₁. ₆alkyl, C₂₋₆alkenyl, C₂₋₆alkynyl, C₃₋₆cycloalkyl, C₂₋₉heterocycloalkyl, C₆₋₁₀aryl, and C₁_ ₉heteroaryl are optionally sub stituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋ ₁₀aryl, and C₁₋₉heteroaryl;
each R⁶ is independently selected from hydrogen, C₁₋₆alkyl, and C₁₋₆haloalkyl; and each R⁷ is independently selected C₁₋₆alkyl, C₂₋₆alkenyl, C₂₋₆alkynyl, C₃₋₆cycloalkyl, C₂₋ ₉heterocycloalkyl, C₆₋₁₀aryl, and C₁₋₉heteroaryl, wherein C₁-₆alkyl, C₂₋₆alkenyl, C₂₋ ₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋ ₉heteroaryl;
each R⁸ is independently selected from hydrogen, halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and
each R⁹ is independently selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and n is 0, 1, 2, 3, or 4.

In some embodiments is a compound of Formula (I) or (I′), or a pharmaceutically acceptable salt or solvate thereof, wherein R ¹ is

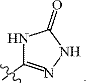

In some embodiments is a compound of Formula (I) or (I′), or a pharmaceutically acceptable salt or solvate thereof, wherein R¹is

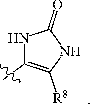

In some embodiments is a compound of Formula (I) or (I′), or a pharmaceutically acceptable salt or solvate thereof, wherein R₁ is

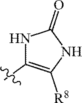

and R⁸ is hydrogen. In some embodiments is a compound of Formula (I) or (I′), or a pharmaceutically acceptable salt or solvate thereof, wherein R₁ is

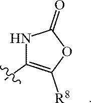

In some embodiments is a compound of Formula (I) or (I′), or a pharmaceutically acceptable salt or solvate thereof, wherein R₁ is

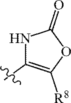

and R⁸ is hydrogen. In some embodiments is a compound of Formula (I) or (I′), or a pharmaceutically acceptable salt or solvate thereof, wherein R¹ is

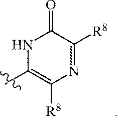

In some embodiments is a compound of Formula (I) or (I′), or a pharmaceutically acceptable salt or solvate thereof, wherein R₁ is

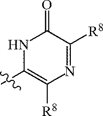

and each R⁸ is hydrogen. In some embodiments is a compound of Formula (I′), or a pharmaceutically acceptable salt or solvate thereof, wherein R¹ is

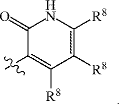

In some embodiments is a compound of Formula (I′), or a pharmaceutically acceptable salt or solvate thereof, wherein R₁ is

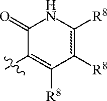

and each R⁸ is hydrogen.. In some embodiments is a compound of Formula (I) or (I′), or a pharmaceutically acceptable salt or solvate thereof, wherein R¹ is

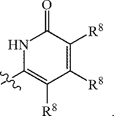

In some embodiments is a compound of Formula (I) or (I′), or a pharmaceutically acceptable salt or solvate thereof, wherein R₁ is

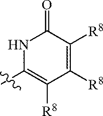

and each R⁸ is hydrogen. In some embodiments is a compound of Formula (I) or (I′), or a pharmaceutically acceptable salt or solvate thereof, wherein R₁ is

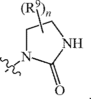

In some embodiments is a compound of Formula (I) or (I′), or a pharmaceutically acceptable salt or solvate thereof, wherein R¹ is

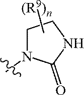

and n is 0. In some embodiments is a compound of Formula (I) or (I′), or a pharmaceutically acceptable salt or solvate thereof, wherein R₁ is

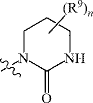

In some embodiments is a compound of Formula (I) or (I′), or a pharmaceutically acceptable salt or solvate thereof, wherein R₁ is

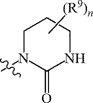

and n is 0. In some embodiments is a compound of Formula (I) or (I′), or a pharmaceutically acceptable salt or solvate thereof, wherein R₁ is

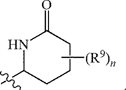

In some embodiments is a compound of Formula (I) or (I′), or a pharmaceutically acceptable salt or solvate thereof, wherein R₁ is

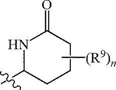

and n is 0.

In some embodiments is a compound of Formula (I) or (I′), or a pharmaceutically acceptable salt or solvate thereof, wherein R² is selected from C₁₋₆alkyl, -C₁₋₆alkyl-C₃₋₆cycloalkyl, -C₁₋₆alkyl-C₆₋₁₀aryl, and -C₁₋₆alkyl-C₁₋₉heteroaryl, wherein C₁₋₆alkyl, -C₁₋₆alkyl-C₃₋₆cycloalkyl, -C₁₋₆alkyl-C₆₋₁₀aryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is selected from C₁₋ ₆alkyl, C₃₋₆cycloalkyl, -C₁₋₆alkyl-C₃₋₆cycloalkyl, -C₁₋₆alkyl-C₆₋₁₀aryl, and -C₁₋₆alkyl-C₁₋₉heteroaryl, wherein C₁₋₆alkyl, C₃₋₆cycloalkyl, -C₁₋₆alkyl-C₃₋₆cy cloalkyl, -C₁₋₆alkyl-C₆₋₁₀aryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is C₁₋₆alkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted C₁₋₆alkyl.

In some embodiments is a compound of Formula (I) or (I′), or a pharmaceutically acceptable salt or solvate thereof, wherein R² is -C₁₋₆alkyl-C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted -C₁₋₆alkyl-C₃₋₆cycloalkyl.

In some embodiments is a compound of Formula (I) or (I′), or a pharmaceutically acceptable salt or solvate thereof, wherein R² is C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted C₃₋₆cycloalkyl.

In some embodiments is a compound of Formula (I) or (I′), or a pharmaceutically acceptable salt or solvate thereof, wherein R² is -C₁₋₆alkyl-phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted -C₁₋₆alkyl-phenyl.

In some embodiments is a compound of Formula (I) or (I′), or a pharmaceutically acceptable salt or solvate thereof, wherein R² is -C₁₋₆alkyl-C₁₋₉heteroaryl optionally substituted with one, two, three, or four groups selected from halogen, C ₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments is a compound of Formula (I) or (I′), or a pharmaceutically acceptable salt or solvate thereof, wherein R² is unsubstituted -C₁₋₆alkyl-C₁₋₉heteroaryl.

In some embodiments, provided herein is a compound of Formula (I) or (I′), or a pharmaceutically acceptable salt or solvate thereof, wherein R² is —CH₃, —CH₂CH₃, —C(H)(CH₃)₂, —CH₂C(H)(CH₃)₂

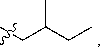

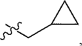

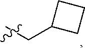

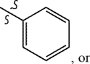

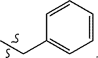

In some embodiments, provided herein is a compound of Formula (I) or (I′), or a pharmaceutically acceptable salt or solvate thereof, wherein R² is —CH₃, —CH₂CH₃, —C(H)(CH₃)₂, —CH₂C(H)(CH₃)₂,

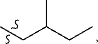

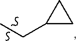

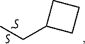

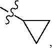

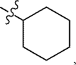

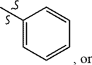

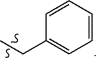

In some embodiments, provided herein is a compound of Formula (I) or (I′), or a pharmaceutically acceptable salt or solvate thereof, wherein R² is —CH₃. In some embodiments, provided herein is a compound of Formula (I) or (I′), or a pharmaceutically acceptable salt or solvate thereof, wherein R² is —CH₂CH₃. In some embodiments, provided herein is a compound of Formula (I) or (I′), or a pharmaceutically acceptable salt or solvate thereof, wherein R²is —C(H)(CH₃)₂. In some embodiments, provided herein is a compound of Formula (I) or (I′), or a pharmaceutically acceptable salt or solvate thereof, wherein R² is —CH₂C(H)(CH₃)₂. In some embodiments, provided herein is a compound of Formula (I) or (I′), or a pharmaceutically acceptable salt or solvate thereof, wherein R² is

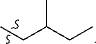

In some embodiments, provided herein is a compound of Formula (I) or (I′), or a pharmaceutically acceptable salt or solvate thereof, wherein R² is

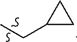

In some embodiments, provided herein is a compound of Formula (I) or (I′), or a pharmaceutically acceptable salt or solvate thereof, wherein R² is

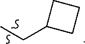

In some embodiments, provided herein is a compound of Formula (I) or (I′), or a pharmaceutically acceptable salt or solvate thereof, wherein R² is

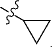

In some embodiments, provided herein is a compound of Formula (I) or (I′), or a pharmaceutically acceptable salt or solvate thereof, wherein R² is

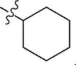

In some embodiments, provided herein is a compound of Formula (I) or (I′), or a pharmaceutically acceptable salt or solvate thereof, wherein R² is

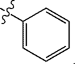

In some embodiments, provided herein is a compound of Formula (I) or (I′), or a pharmaceutically acceptable salt or solvate thereof, wherein R² is

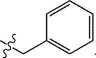

In some embodiments, provided herein is a compound of Formula (I) or (I′), or a pharmaceutically acceptable salt or solvate thereof, wherein R³ is C₁₋₉heteroaryl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R³ is unsubstituted C₁₋₉heteroaryl. In some embodiments, provided herein is a compound of Formula (I) or (I′), or a pharmaceutically acceptable salt or solvate thereof, wherein R³ is

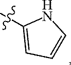

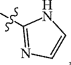

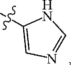

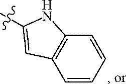

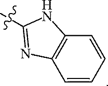

In some embodiments, provided herein is a compound of Formula (I) or (I′), or a pharmaceutically acceptable salt or solvate thereof, wherein R³ is

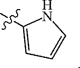

In some embodiments, solvate thereof, wherein R³ is

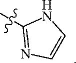

In some embodiments, provided herein is a compound of Formula (I) or (I′), or a pharmaceutically acceptable salt or solvate thereof, wherein R³ is

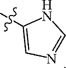

acceptable salt or solvate thereof, wherein R³ is

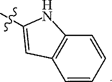

In some embodiments, provided wherein R³ is

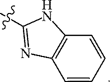

In some embodiments, provided herein is a compound of Formula (I) or (I′), or a pharmaceutically acceptable salt or solvate thereof, wherein R³ is C₆₋₁₀aryl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)_{N}(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R³ is unsubstituted C₆₋₁₀aryl. In some embodiments, provided herein is a compound of Formula (I) or (I′), or a pharmaceutically acceptable salt or solvate thereof, wherein R³ is naphthyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R³ is unsubstituted naphthyl. In some embodiments, provided herein is a compound of Formula (I) provided herein is a compound of Formula (I) or (I′), or a pharmaceutically acceptable salt or herein is a compound of Formula (I) or (I′), or a pharmaceutically acceptable salt or solvate thereof,

In some embodiments, provided herein is a compound of Formula (I) or (I′), or a pharmaceutically or (I′), or a pharmaceutically acceptable salt or solvate thereof, wherein R³ is phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R³ is unsubstituted phenyl.

In some embodiments, provided herein is a compound of Formula (I) or (I′), or a pharmaceutically acceptable salt or solvate thereof, wherein R³ is C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R³ is unsubstituted C₃₋₆cycloalkyl.

In some embodiments, provided herein is a compound of Formula (I) or (I′), or a pharmaceutically acceptable salt or solvate thereof, wherein R³ is C₂₋₉heterocycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R³ is unsubstituted C₂₋₉heterocycloalkyl.

In some embodiments, provided herein is a compound of Formula (I) or (I′), or a pharmaceutically acceptable salt or solvate thereof, wherein R⁴ is selected from C₁₋₆alkyl, C₃₋ ₆cycloalkyl, -C₁₋₆alkyl-C₃₋₆cycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₃₋₆cycloalkyl, -C₁₋₆alkyl-C₃₋₆cycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R⁴ is selected from C₃₋₆cycloalkyl, -C₁₋₆alkyl-C₃₋₆cycloalkyl, C₆₋₁₀aryl, and -C₁₋₆alkyl-C₆₋₁₀aryl, wherein C₃₋₆cycloalkyl, -C₁₋₆alkyl-C₃₋₆cycloalkyl, C₆₋₁₀aryl, and -C₁₋₆alkyl-C₆₋₁₀aryl are optionally substituted with one, two, three, or four groups selected from halogen, C₁₋₆alkyl, C₁₋ ₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R⁴ is C₃₋₆cycloalkyl optionally substituted with one, two, three, or four groups selected from halogen, C ₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R⁴ is unsubstituted C₃₋₆cycloalkyl.

In some embodiments, provided herein is a compound of Formula (I) or (I′), or a pharmaceutically acceptable salt or solvate thereof, wherein R⁴ is -C₁₋₆alkyl-C₃₋₆cycloalkyl optionally substituted with one, two, three, or four groups selected from halogen, C₁₋₆alkyl, C₁₋ ₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R⁴ is unsubstituted -C₁₋₆alkyl-C₃₋₆cycloalkyl.

In some embodiments, provided herein is a compound of Formula (I) or (I′), or a pharmaceutically acceptable salt or solvate thereof, wherein R⁴ is phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R⁴ is unsubstituted phenyl.

In some embodiments, provided herein is a compound of Formula (I) or (I′), or a pharmaceutically acceptable salt or solvate thereof, wherein R⁴ is -C₁₋₆alkyl-phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—. In some embodiments, provided herein is a compound of Formula (I) or (I′), or a pharmaceutically acceptable salt or solvate thereof, wherein R⁴ is unsubstituted -C₁₋₆alkyl-phenyl.

In some embodiments, provided herein is a compound of Formula (I) or (I′), or a pharmaceutically acceptable salt or solvate thereof, wherein R⁴ is

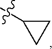

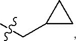

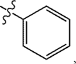

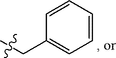

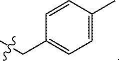

In some embodiments, provided herein is a compound of Formula (I) or (I′), or a pharmaceutically acceptable salt or solvate thereof, wherein R⁴ is

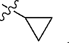

In some embodiments, provided herein is a compound of Formula (I) or (I′), or a pharmaceutically acceptable salt or solvate thereof, wherein R⁴ is

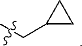

In some embodiments, provided herein is a compound of Formula (I) or (I′), or a pharmaceutically acceptable salt or solvate thereof, wherein R⁴ is

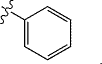

In some embodiments, provided herein is a compound of Formula (I) or (I′), or a pharmaceutically acceptable salt or solvate thereof, wherein R⁴ is

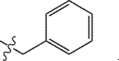

In someembodiments, provided herein is a compound of Formula (I) or (I′), or a pharmaceutically acceptable salt or solvate thereof, wherein R⁴ is

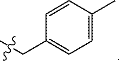

In some embodiments, provided herein is a compound selected from:

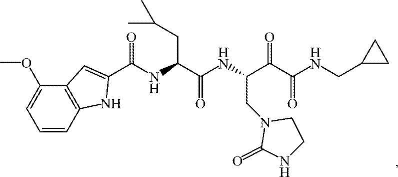

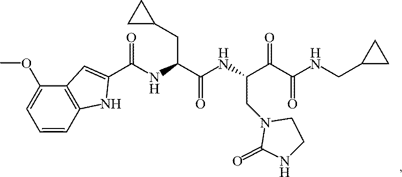

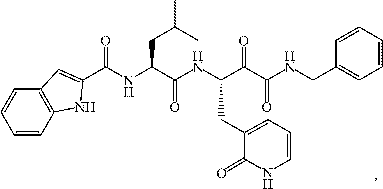

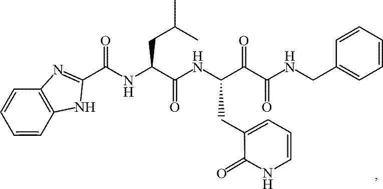

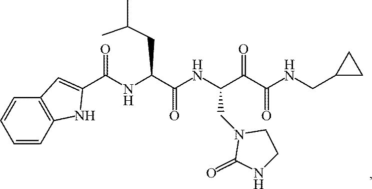

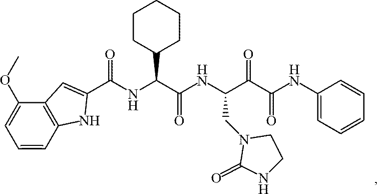

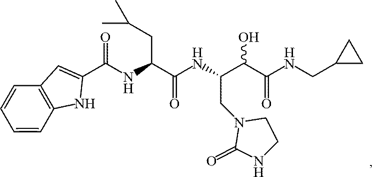

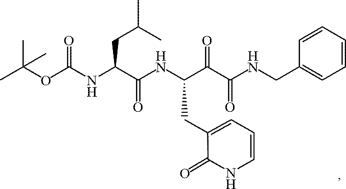

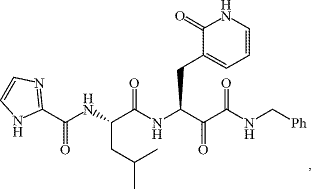

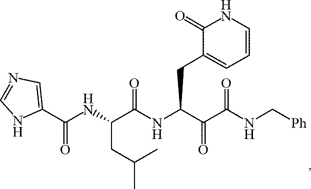

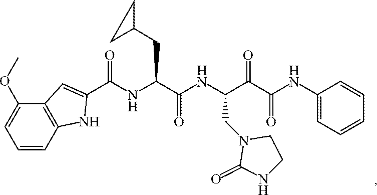

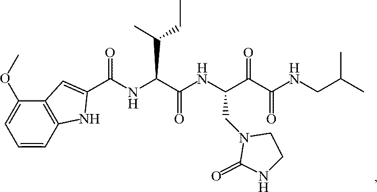

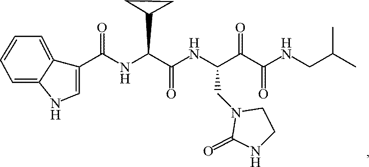

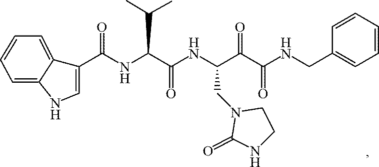

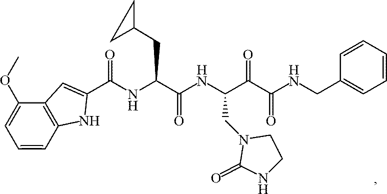

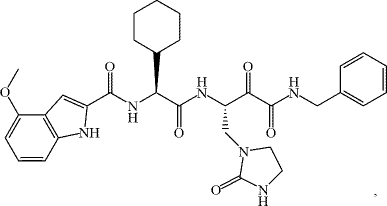

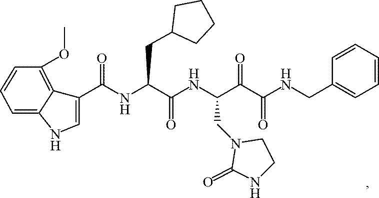

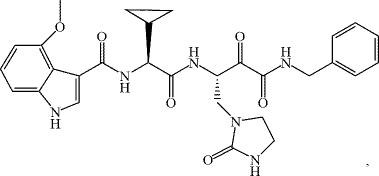

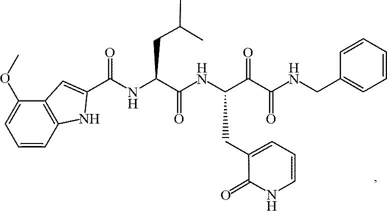

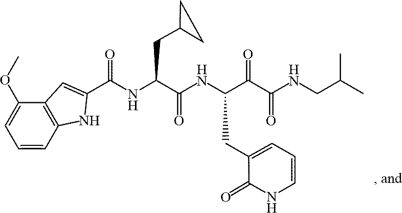

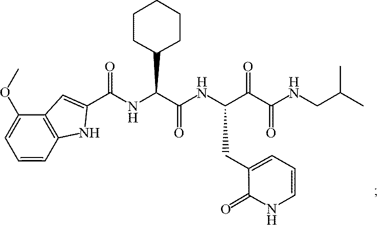

; or a pharmaceutically acceptable salt or solvate thereof.

In some embodiments, provided herein is a compound selected from:

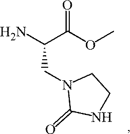

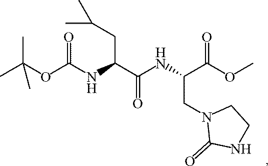

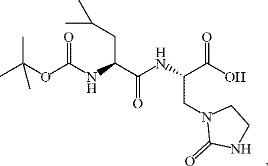

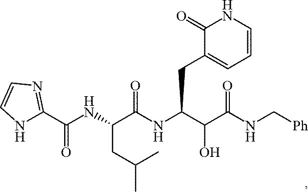

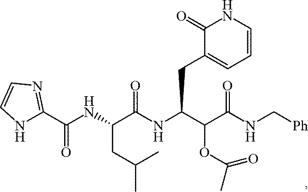

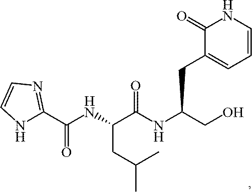

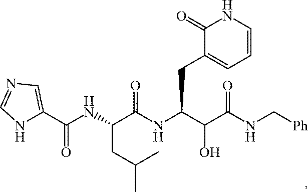

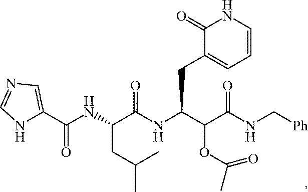

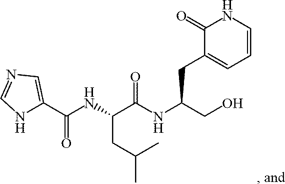

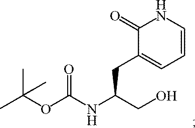

; or a pharmaceutically acceptable salt or solvate thereof.

In some embodiments, provided herein is a compound selected from:

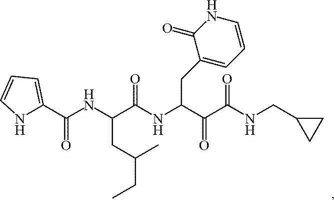

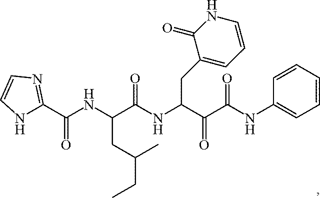

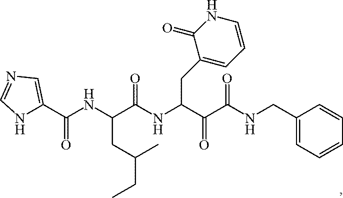

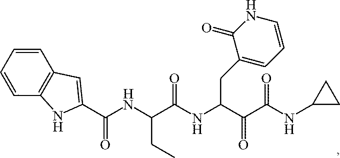

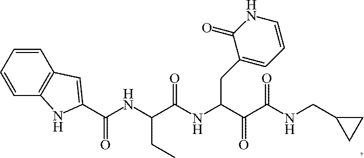

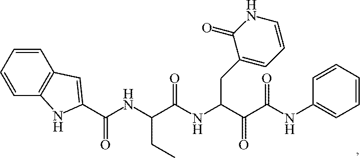

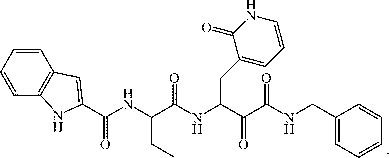

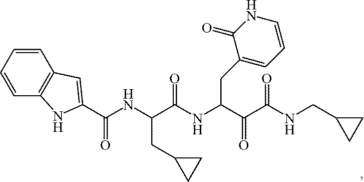

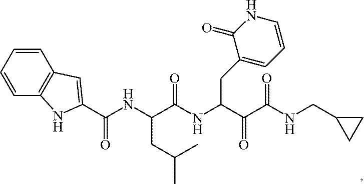

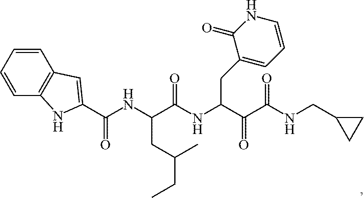

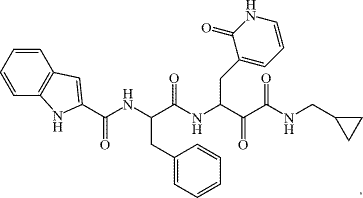

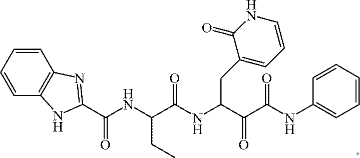

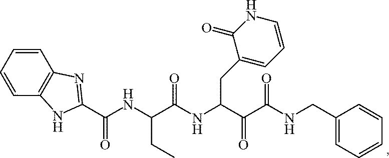

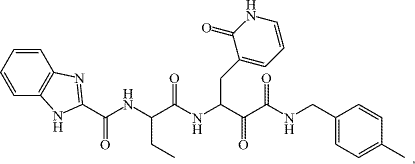

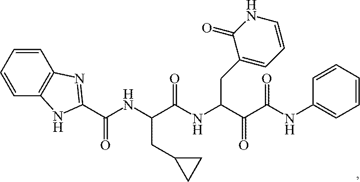

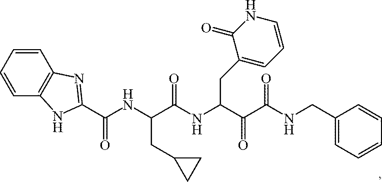

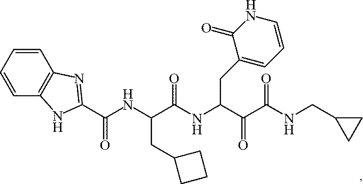

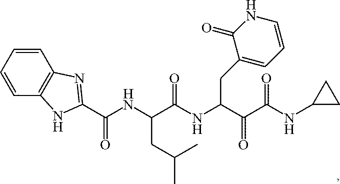

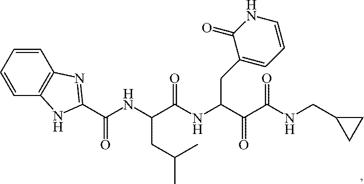

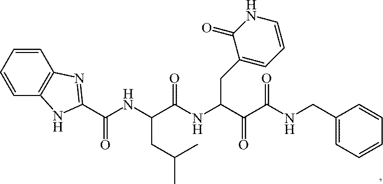

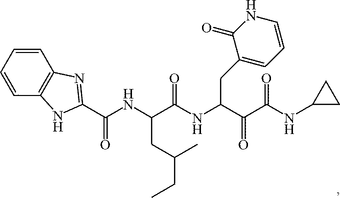

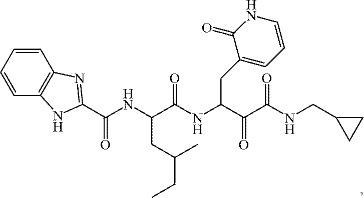

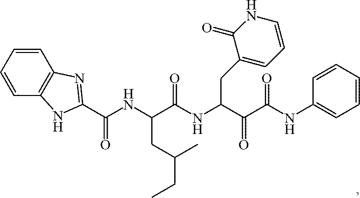

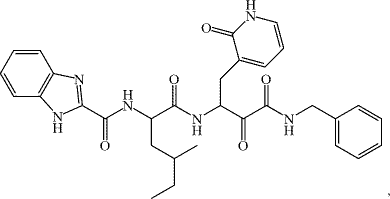

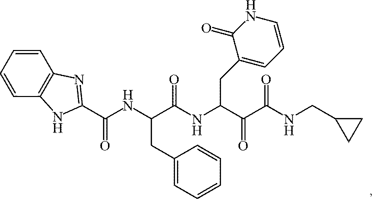

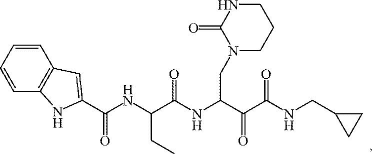

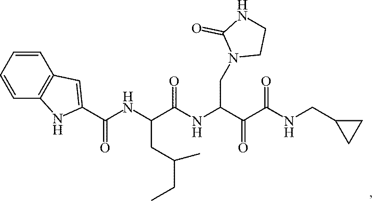

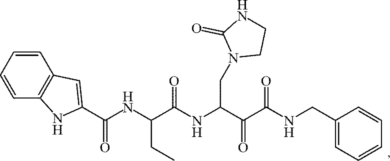

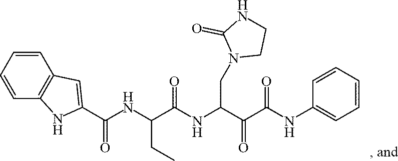

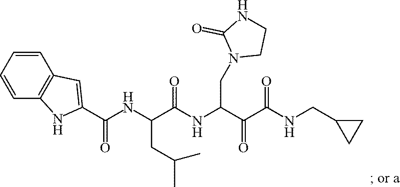

pharmaceutically acceptable salt or solvate thereof.

Any combination of the groups described above for the various variables is contemplated herein. Throughout the specification, groups and substituents thereof can be chosen by one skilled in the field to provide stable moieties and compounds.

In some embodiments, the therapeutic agent(s) (e.g. compound of Formula (I) or (I′)) is present in the pharmaceutical composition as a pharmaceutically acceptable salt. In some embodiments, any compound described above is suitable for any method or composition described herein.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁷O, ¹⁸O, ³¹P, ³²P, ³⁵S, ¹⁸F, and ³⁶Cl, respectively. Compounds described herein, and pharmaceutically acceptable salts, esters, solvate, hydrates, or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991), Larock’s Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. As a guide the following synthetic methods may be utilized:

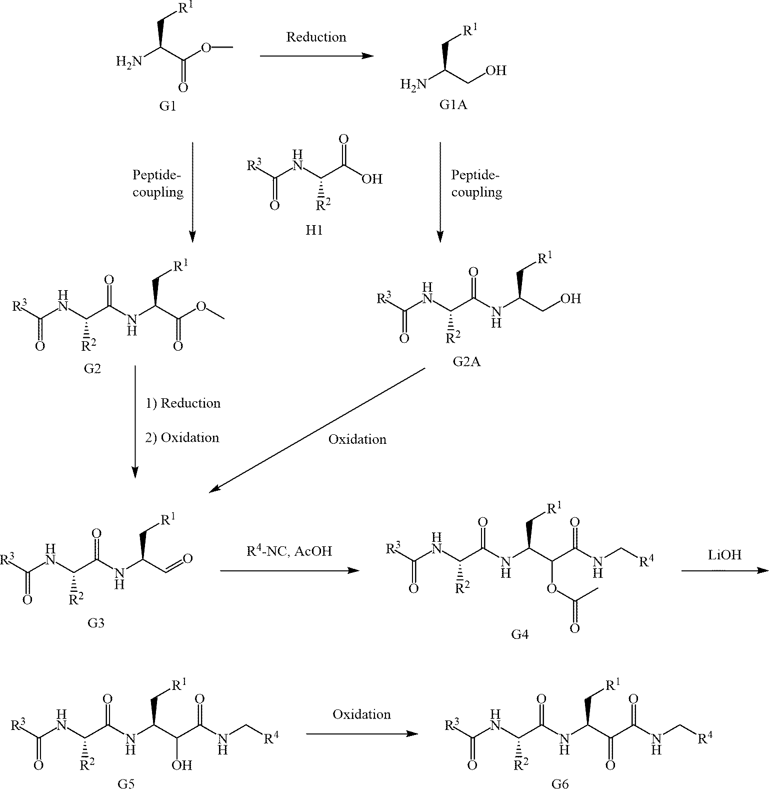

Amino acid analog G1 is coupled with peptide H1 in the presence of an activator such as EDC-Cl (1-ethyl-3-(3-dimethylaminopropyl)carbodiimide), an additive to suppress isomerization such as HOBt (hydroxybenzotriazole), a base such as triethylamine, and a suitable organic solvent such as dimethylformamide.

The resulting dipeptide G2 is then reduced to a primary alcohol using a reductant (e.g. lithium aluminum hydride) and oxidized to aldehyde G3 in the presence of an oxidant such as a hypervalent iodine compound (e.g. Dess-Martin periodinane), a sulfonium (e.g. Swern oxidation), or hexavalent chromium (e.g. Collins reagent, PDC, or PCC). Alternatively, G1 is first reduced to alcohol G1A and coupled to peptide H1 using the above described conditions, and then oxidized to aldehyde G3. Formation of acetate G4 proceeds through exposure of aldehyde G3 to R⁴-substituted isocyanide in the presence of acetic acid. Exposure of acetate G4 to aqueous hydroxide (e.g. LiOH/H₂O) and oxidation of resulting hydroxyamide G5 with a suitable oxidant (e.g. PCC, Dess-Martin periodinane, Swern oxidation, TEMPO oxidation) provides the target α-ketoamide G6.

### Use of Protecting Groups

In the reactions described, it may be necessary to protect reactive functional groups, for example hydroxy, amino, imino, thio or carboxy groups, where these are de sired in the final product, in order to avoid their unwanted participation in reactions. Protecting groups are used to block some or all of the reactive moieties and prevent such groups from participating in chemical reactions until the protective group is removed. It is preferred that each protective group be removable by a different means. Protective groups that are cleaved under totally disparate reaction conditions fulfill the requirement of differential removal.

Protective groups can be removed by acid, base, reducing conditions (such as, for example, hydrogenolysis), and/or oxidative conditions. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, which include conversion to alkyl esters, or they may be blocked with oxidatively -removable protective groups such as 2,4-dimethoxybenzyl, while co-existing amino groups may be blocked with fluoride labile silyl carbamates.

Allyl blocking groups are useful in the presence of acid- and base- protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

### Methods of Treatment

In some embodiments is a method of treating a coronavirus infection in an individual in need thereof, comprising administering to the individual a therapeutically effective amount of a compound Formula (I) or (I′), or a pharmaceutically acceptable salt or solvate thereof. In some embodiments is a method of treating a coronavirus infection in an individual in need thereof, comprising administering to the individual a therapeutically effective amount of a compound Formula (I) or (I′), or a pharmaceutically acceptable salt or solvate thereof, wherein the coronavirus is severe acute respiratory syndrome coronavirus 2 (SARS-CoV-2). In some embodiments is a method of treating COVID-19 in an individual in need thereof, comprising administering to the individual a therapeutically effective amount of a compound Formula (I) or (I′), or a pharmaceutically acceptable salt or solvate thereof. In some embodiments is a method of treating COVID-19 in an individual in need thereof, comprising administering to the individual a therapeutically effective amount of a compound Formula (I) or (I′), or a pharmaceutically acceptable salt or solvate thereof. In some embodiments is a method of treating severe acute respiratory syndrome (SARS) in an individual in need thereof, comprising administering to the individual a therapeutically effective amount of a compound Formula (I) or (I′), or a pharmaceutically acceptable salt or solvate thereof. In some embodiments is a method of treating Middle East respiratory syndrome (MERS) in an individual in need thereof, comprising administering to the individual a therapeutically effective amount of a compound Formula (I) or (I′), or a pharmaceutically acceptable salt or solvate thereof.

### Pharmaceutical Compositions and Methods of Administration

Mpro cysteine protease inhibitors described herein are administered to subjects in a biologically compatible form suitable for administration to treat or prevent diseases, disorders or conditions. Administration of Mpro cysteine protease inhibitors as described herein can be in any pharmacological form including a therapeutically effective amount of an Mpro cysteine protease inhibitor alone or in combination with a pharmaceutically acceptable carrier.

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

In some embodiments is a pharmaceutical composition comprising a pharmaceutically acceptable carrier and a compound of Formula (I′), or a pharmaceutically acceptable salt or solvate thereof. In some embodiments is a pharmaceutical composition consisting essentially of a pharmaceutically acceptable carrier and a compound of Formula (I′), or a pharmaceutically acceptable salt or solvate thereof. In some embodiments is a pharmaceutical composition comprising a pharmaceutically acceptable carrier and a compound of Formula (I), or a pharmaceutically acceptable salt or solvate thereof. In some embodiments is a pharmaceutical composition consisting essentially of a pharmaceutically acceptable carrier and a compound of Formula (I), or a pharmaceutically acceptable salt or solvate thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, topical, buccal, parenteral (e.g., subcutaneous, intramuscular, intradermal, or intravenous), or aerosol administration.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments, Mpro cysteine protease inhibitors described herein are administered to subjects in a biologically compatible form suitable for topical administration to treat or prevent dermal diseases, disorders, or conditions. By “biologically compatible form suitable for topical administration” is meant a form of the Mpro cysteine protease inhibitor to be administered in which any toxic effects are outweighed by the therapeutic effects of the inhibitor. Administration of Mpro cysteine protease inhibitors as described herein can be in any pharmacological form including a therapeutically effective amount of an Mpro cysteine protease inhibitor alone or in combination with a pharmaceutically acceptable carrier.

Topical administration of an Mpro cysteine protease inhibitors may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington’s Pharmaceutical Sciences, 15th Edition (1975) published by Mack Publishing Company.

Dermal or skin patches are another method for transdermal delivery of the therapeutic or pharmaceutical compositions described herein. Patches can provide an absorption enhancer such as DMSO to increase the absorption of the compounds. Patches can include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin. The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

In one embodiment, the topical composition may, for example, take the form of hydrogel based on polyacrylic acid or polyacrylamide; as an ointment, for example with polyethyleneglycol (PEG) as the carrier, like the standard ointment DAB 8 (50% PEG 300, 50% PEG 1500); or as an emulsion, especially a microemulsion based on water-in-oil or oil-in-water, optionally with added liposomes. Suitable permeation accelerators (entraining agents) include sulphoxide derivative s such as dimethylsulphoxide (DMSO) or decylmethylsulphoxide (decyl-MSO) and transcutol (diethyleneglycolmonoethylether) or cyclodextrin; as well as pyrrolidones, for example 2-pyrrolidone, N-methyl-2-pyrrolidone, 2-pyrrolidone-5-carboxylic acid, or the biodegradable N-(2-hydroxyethyl)-2-pyrrolidone and the fatty acid esters thereof; urea derivatives such as dodecylurea, 1,3-didodecylurea, and 1,3-diphenylurea; and terpenes, for example D-limonene, menthone, a-terpinol, carvol, limonene oxide, or 1,8-cineol.

Ointments, pastes, creams and gels also can contain excipients, such as starch, tragacanth, cellulose derivatives, polyethylene glycols, silicones, bentonites, silicic acid, and talc, or mixtures thereof. Powders and sprays also can contain excipients such as lactose, talc, silicic acid, aluminum hydroxide, calcium silicates and polyamide powder, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such a chlorofluorohydrocarbons and volatile unsubstituted hydrocarbons, such as butane and propane.

The carrier can also contain other pharmaceutically-acceptable excipients for modifying or maintaining the pH, osmolarity, viscosity, clarity, color, sterility, stability, rate of dissolution, or odor of the formulation. The anti-skin aging compositions can also further comprise antioxidants, sun screens, natural retinoids (e.g., retinol), and other additives comm only found in skin treatment compositions.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragees, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In some embodiments, suspensions, in addition to the subject composition, contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In some embodiments, powders and sprays contain, in addition to a subject composition, excipients such as lactose, talc, silicic acid, aluminum hydroxide, calcium silicates, and polyamide powder, or mixtures of these substances. In some embodiments, sprays additionally contain customary propellants, such as chlorofluorohydrocarbons and volatile unsubstituted hydrocarbons, such as butane and propane.

Compositions and compounds disclosed herein alternatively are administered by aerosol. This is accomplished by preparing an aqueous aerosol, liposomal preparation, or solid particles containing the compound. In some embodiments, a non-aqueous (e.g., fluorocarbon propellant) suspension is used. In some embodiments, sonic nebulizers are used because they minimize exposing the agent to shear, which results in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in some embodiments, contain antioxidants, buffers, bacteriostats, solutes which render the formulation isotonic with the blood of the intended recipient, or suspending or thickening agents.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants

The dose of the composition comprising at least one compound described herein differs, depending upon the patient’s (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient’s disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the Mpro cysteine protease inhibitor and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the Mpro cysteine protease inhibitor activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient’s symptoms, and the chosen route of administration.

Toxicity and therapeutic efficacy of such Mpro cysteine protease inhibitors can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀ /ED₅₀. Mpro cysteine protease inhibitors that exhibit large therapeutic indices are preferred. While Mpro cysteine protease inhibitors that exhibit toxic side effects may be used, care should be taken to design a delivery system that targets such inhibitors to the site of affected tissue in order to minimize potential damage to uninfected cells and, thereby, reduce side effects.

The data obtained from the cell culture assays and animal studies can be used in formulating a range of dosage for use in humans. The dosage of such Mpro cysteine protease inhibitors lies preferably within a range of circulating concentrations that include the ED₅₀ with little or no toxicity. The dosage may vary within this range depending upon the dosage form employed and the route of administration utilized. For any Mpro cysteine protease inhibitor used in a method described herein, the therapeutically effective dose can be estimated initially from cell culture assays. A dose may be formulated in animal models to achieve a circulating plasma concentration range that includes the IC₅₀ (i.e., the concentration of Mpro cysteine protease inhibitor that achieves a half-maximal inhibition of symptoms) as determined in cell culture. Such information can be used to more accurately determine useful doses in humans. Levels in plasma may be measured, for example, by high performance liquid chromatography.

### EXAMPLES

The following examples are offered for purposes of illustration and are not intended to limit the scope of the claims provided herein. All literature citations in these examples and throughout this specification are incorporated herein by references for all legal purposes to be served thereby. The starting materials and reagents used for the synthesis of the compounds described herein may be synthesized or can be obtained from commercial sources, such as, but not limited to, Sigma-Aldrich, Acros Organics, Fluka, and Fischer Scientific.

Standard abbreviations and acronyms as defined in J. Org. Chem. 2007 72(1): 23A-24A are used herein. Other abbreviations and acronyms used herein are as follows:

| | |
|---|---|
| AcOH | acetic acid |
| DCM | dichloromethane |
| DMF | dimethylformamide |
| DMP | Dess-Martin periodinane |
| EtOAc | ethyl acetate |
| EtOH | ethanol |
| eq | equivalent |
| HATU | (1-[bis(dimethylamino)methylene]-1H-1,2,3-triazolo[4,5-b]pyridinium 3-oxide hexafluorophosphate |
| HBTU | N,N,N′,N′-tetramethyl-O-(1H-benzotriazol-1-yl)uronium hexafluorophosphate |
| HOAt | 1-Hydroxy-7-azabenzotriazole |
| LC-MS | liquid chromatography-mass spectrometry |
| MeOH | methanol |
| TEA | triethylamine |
| TFA | trifluoroacetic acid |
| THF | tetrahydrofuran |
| rt | room temperature |

### Example 1: Synthesis of N-((S)-1-(((S)-4-((Cyclopropylmethyl)Amino)-3,4-Dioxo-1-(2-Oxoimidazolidin-1-yl)Butan-2-yl)Amino)-4-Methyl-1-Oxopentan-2-yl)-4-Methoxy-1H-Indole-2-Carboxamide (10)

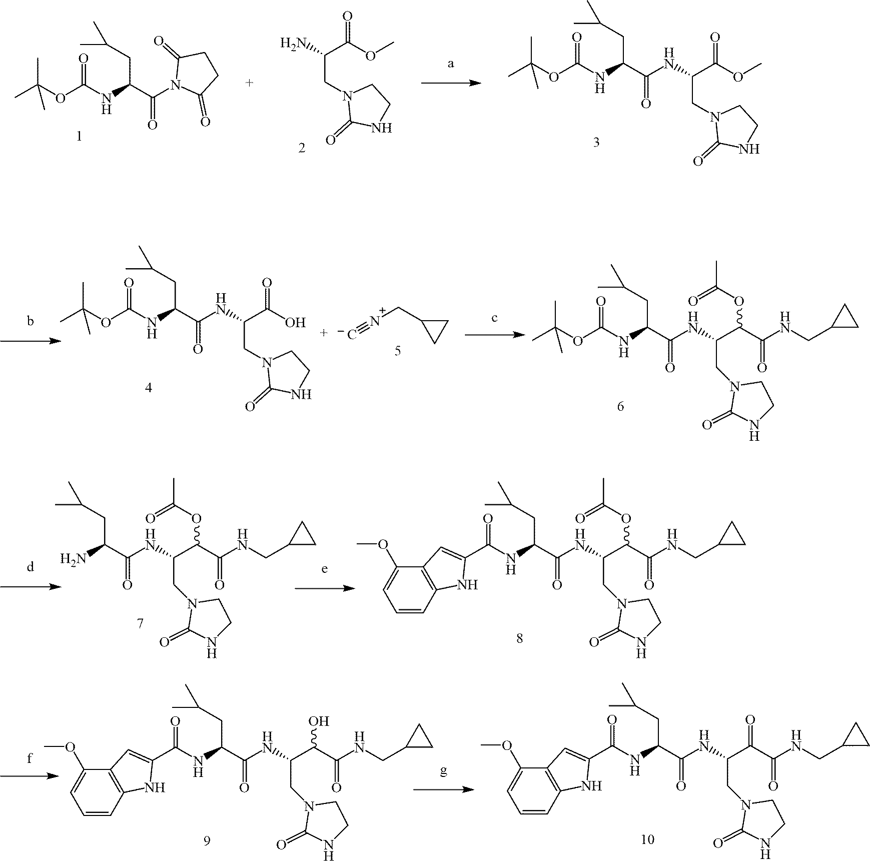

Step a: Compound 2 (1.7 mMol, 280 mg) was dissolved in DMF (5 mL) and Boc-(S)-Leu-OSu (compound 1) (2.04 mMol, 637 mg) and TEA (200 µL) were added at RT and stirred for 16 h. The reaction mixture was concentrated and re-dissolved in DCM and washed with saturatedammonium chloride, dried with sodium sulfate and concentrated. The crude reaction mixture was loaded to a column and eluted with 1-3% methanol: DCM to obtain compound 3 (480 mg, 71%) as a white solid.

Step b: To a stirred solution of compound 3 (1.19 mMol, 480 mg) in THF (10 mL) and ethanol (300 µL) was added slowly lithium borohydride in THF (1.0 mL of a 4 M solution) at RT and stirred for 30 min. The reaction mixture was quenched with a few drops of acetone, diluted with ethyl acetate and then washed with water. The solution was concentrated to give compound 4 (335 mg, 75%) as a white solid. MS(ESI): 373 (MH⁺).

Step c: To a solution of compound 4 (0.89 mM, 335 mg) in 10 mL DCM were added Dess-Martin periodinane (DMP) (457 mg) and NaHCO₃ (100 mg) and stirred at RT. After 10 min 60 µL acetic acid and cyclopropylmethyl isonitrile (compound 5) (100 µL) were added at RT and stirred for 24 h. The reaction mixture was stirred for another 16 h until the starting material disappeared. After filtration, concentration and dissolution in DCM chromatography gave compound 6 (240 mg, 42%) as a slightly yellow solid. MS (ESI): 512 (MH⁺).

Step d: To a solution of compound 6 (0.21 mM, 110 mg) in DCM (3 mL) was added 1 mL TFA and stirred for 1h. The reaction mixture was concentrated and co-evaporated with DCM several times to afford compound 7 which was used in the next step without further purification.

Step e: 4-Methoxy indol-2-carboxylic acid (0.26 mM, 50 mg) and compound 7 (0.21 mM) were dissolved in DMF (3 mL) and HATU (0.27 mM, 102 mg), TEA (200 µL) and HOAt (0.27 mM, 6 mg) were added at RT and stirred for 30 min. The reaction mixture was concentrated and redissolved in DCM and washed with water. The organic solution was dried with sodium sulfate and concentrated. The crude reaction mixture was loaded to a column and eluted with DCM, 1-10 % methanol: DCM. The fractions containing the product were collected to yield compound 8 (58 mg, 47%). MS (ESI): 585 (MH⁺).

Step f: To a solution of compound 8 (0.094 mM, 55 mg) in MeOH (8 mL) were added of a saturated solution of K₂CO₃ (100 µL) at RT and stirred for 30 min. Saturated brine (8 mL) was added and extracted three times with 10 mL of ethyl acetate. The combined organic phases were dried over sodium sulfate and concentrated to yield compound 9 (50 mg, 92%).

Step g: To a solution of compound 9 (0.53 mM, 290 mg) in DCM (20 mL) was added Dess-Martin periodinane (250 mg) at RT and stirred for 1 h. The reaction mixture was concentrated to half of the volume and loaded to a column and eluted with 1-3% methanol : DCM. The fractions containing the product were collected to yield compound 10 (180 mg, 62%) as a gray-white solid. MS (ESI): 541 (MH⁺). ¹H-NMR (400 MHz, CDCl₃): δ 0.16 (m, 2H), 0.45 (m, 2H), 0.88 (m, 7H, 2x CH₃, CH), 1.5-1.9 (m, 3H, CH₂, CH), 3.02-3.20 (m, 4H, 2 x CH₂), 3.22 (m, 1H), 3.45 (m, 1H), 3.56 (m, 1H), 3.72 (m, 1H), 3.85 (s, 3H), 4.78 (m, 1H), 5.42 (m, 1H), 6.40 (d, 1H), 6.41-7.14 (m, 4H), 9.95 (br s, 1H).

### Synthesis of Intermediate Compound 2

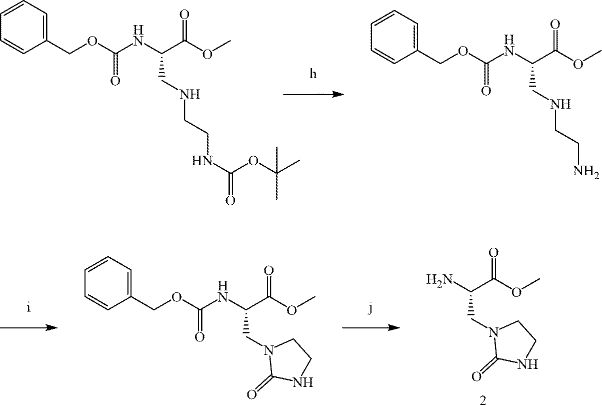

Step h: Methyl (S)-2-(((benzyloxy)carbonyl)amino)-3-((2-((tert-butoxycarbonyl)amino)ethyl)amino)propanoate (prepared as described in J. Org. Chem., 2002, 67, 4017-4029) (34.6 mMol, 13.7 g) was dissolved in DCM (100 mL) and TFA (40 mL) was added at RT and stirred for 1 h. The reaction mixture was concentrated and co-evaporated with DCM several times to remove excess of TFA to afford methyl (S)-3-(2-amino-ethylamino)-2-benzyloxycarbonylamino-propionate as a yellow oil in quantitative yield, which was used without further purification. MS(ESI): 296 (MH⁺).

Step i: Methyl (S)-3-(2-amino-ethylamino)-2-benzyloxycarbonylamino-propionate obtained in step h was dissolved in THF (100 mL) and TEA (6 mL) were added at RT and stirred for 10 min. CDI(34.6 mMol, 5.6 g) in THF (50 mL) were added slowly. The reaction mixture was stirred for 24 h at RT and then concentrated. The residue was dissolved in dichloromethane and purified by chromatography with 2.5% methanol : DCM to afford methyl (S)-2-benzyloxycarbonylamino-3-(2-oxo-imidazolidin-1-yl)-propionate (10 g, 90%) as a colourless oil. MS(ESI): 322 (MH⁺); ¹H-NMR (CDCl₃, 400 MHz): δ 3.24-3.34 (m, 3H, CH₂ + CH₂-H), 3.41-3.54 (m, 3H, CH₂ + CH₂-H), 3.68 (s, 3H, OCH₃), 4.41 (m, 1H, CH), 4.91 (br s, 1H, NH), 5.03 (s, 2H, CH₂-Ph), 6.05 (d, 1H, J=7.6 Hz, NH), 7.20-7.28 (m, 5H).

Step J: Methyl (S)-2-benzyloxycarbonylamino-3-(2-oxo-imidazolidin-1-yl)-propionate (31.1 mMol, 10.0 g) was dissolved in methanol (100 mL) and hydrogenated using 2 g ofPd/C. The reaction mixture was stirred for 8 h at RT. The mixture was filtered and evaporated to afford compound 2 (5 g, 86%) as a clear oil.

### Example 2: Synthesis of N-((S)-3-Cyclopropyl-1-(((S)-4-((Cyclopropylmethyl)Amino)-3,4-Dioxo-1-(2-Oxoimidazolidin-1-yl)Butan-2-yl)Amino)-1-Oxopropan-2-yl)-4-Methoxy-1H-Indole-2-Carboxamide (12)

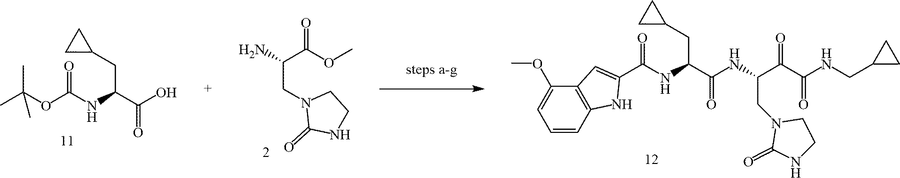

N-((S)-3-Cyclopropyl-1-(((S)-4-((cyclopropylmethyl)amino)-3,4-dioxo-1-(2-oxoimidazolidin-1-yl)butan-2-yl)amino)-1-oxopropan-2-yl)-4-methoxy-1H-indole-2-carboxamide (compound 12) was prepared as described in Example 1, steps a-g, by replacing Boc-(S)-Leu-OSu (compound 1) with (S)-2-((tert-butoxycarbonyl)amino)-3-cyclopropylpropanoic acid (compound 11). Compound 12 MS(ESI): 539 (MH⁺).

### Example 3: Synthesis of N-((S)-1-(((S)-4-(Benzylamino)-3,4-Dioxo-1-(2-Oxo-1,2-Dihydropyridin-3-yl)Butan-2-yl)Amino)-4-Methyl-1-Oxopentan-2-yl)-1H-Indole-2-Carboxamide (22)

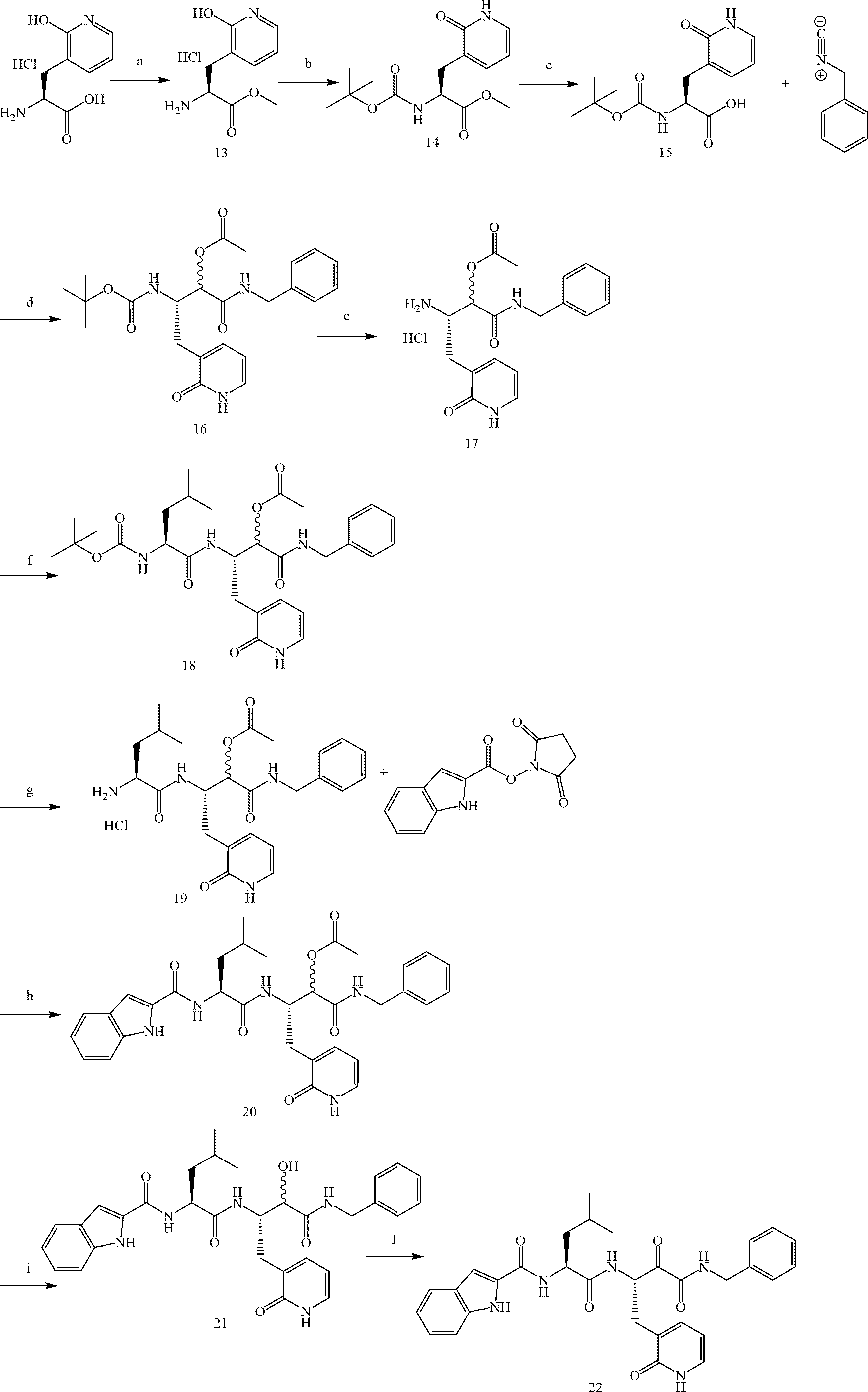

Step a: At 0° C., acetyl chloride (10 mL) was added slowly to methanol (120 mL) and stirred for 30 min. Then commercially available 2S-amino-3-(2-hydroxy-pyridin-3-yl)-propionic acid (0.45 mMol, 500 mg) was added in one portion, stirred at 0° C. for 1h, and then stirred at RT for 12 h. The reaction mixture was concentrated and co-evaporated with toluene, ethyl acetate and then several times with DCM to afford compound 13 as a white foam. MS (ESI): 197 (MH⁺).

Step b: To a solution of compound 13 (2.28 mM) in DMF (70 mL) were added Boc₂O (4.6 mM, 1.0 g) and TEA (3 mL) at RT and the mixture was stirred for 3 h. The reaction mixture was concentrated, re-dissolved in DCM and washed with saturated ammonium chloride. The combined organic phases were dried over sodium sulfate and concentrated. The residue was purified by column chromatography using 3% methanol: DCM to afford compound 14 (550 mg, 81%) as a white solid. MS (ESI): 297 (MH⁺).

Step c: To a solution of compound 14 (1.8 mM, 550 mg) in THF (10 mL) and ethanol (0.5 mL) were added slowly a 4 Mlithium borohydride solution in THF (1.5 mL) at RT. The reaction mixture was stirred for 30 min and quenched with a few drops of acetone and diluted with ethyl acetate. The organic solution was washed with water, dried, and after evaporation of the solvent gave compound 15 (350 mg, 72%) as a white solid. MS (ESI): 269 (MH⁺).

Step d: To a solution of compound 15 (1.3 mM, 350 mg) in a mixture of THF: DCM (1:1) (16 mL) were added DMP (593 mg) and NaHCO₃ (177 mg). The solution was stirred for 10 min. Then acetic acid (90 µL) and benzyl isonitrile (180 µL) were added at RT and stirred for 24 h. The reaction mixture was filtered, concentrated, re-dissolved in DCM and loaded to a column. Elution with 1-3% methanol: DCM gave compound 16 (240 mg, 42%) as a light yellow solid.

Step e: To a solution of compound 16 (0.112 mM, 50 mg) in dioxane (2 mL) was added 0.5 mL 4 M HCl solution in dioxane and stirred for 1.5 h. The reaction mixture was concentrated and co-evaporated with DCM several times to afford compound 17 as a white solid, which was used without further purification in the next step. MS (ESI): 344 (MH⁺).

Step f: To a solution of compound 17 (0.112 mM) in DMF (3 mL) were added TEA (0.2 mL) and Boc-L-Leu-OSu (0.145 mM, 48 mg). The reaction mixture was stirred at RT for 16 h. The reaction mixture was concentrated and the residue purified by column chromatography using 1-5 % MeOH : DCM to afford compound 18 (40 mg, 64%). MS (ESI): 557 (MH⁺).

Step g: To a solution of compound 18 (0.153 mM, 85 mg) in dioxane (3 mL) was added a 4 M HCl solution in dioxane (0.65 mL). The reaction mixture was stirred at RT for 16 h. The reaction mixture was concentrated and dried for one hour in vacuo to afford compound 19, which was used in the next step without further purification. MS (ESI): 457 (MH⁺).

Step h: To a solution of compound 19 (0.153 mM) in DMF (3 mL) was added 2,5-dioxopyrrolidin-1-yl 1H-indole-2-carboxylate (1.5 eq., 60 mg) followed by DIPEA (0.5 mL) at RT. The reaction mixture was stirred at RT for 16 h and then concentrated. The residue was purified by column chromatography eluting with 1% (500 mL) and 6% MeOH: DCM (500 mL) to afford compound 20 (60 mg, 65%) as a white solid. MS (ESI): 600 (MH⁺).

Step i: To a solution of compound 20 (0.058 mM, 35 mg) in MeOH (3 mL) was added a saturated solution of K₂CO₃ (25 µL) at RT. Brine (3 mL) was added and the mixture was extracted with 3 mL of DCM (5-times). The combined organic phases were dried over sodium sulfate and concentrated. A white solid was obtained. The mixture was dissolved in DCM and a few drops of methanol were added. The undissolved residue was filtered and the filter paper was washed with DCM. The solution was concentrated to afford compound 21 (28 mg, 86%). MS (ESI): 558 (MH⁺).

Step j: To a solution of compound 21 (0.05 mM, 28 mg) in DCM (6 mL) was added DMP (25 mg) and sodium hydrogen carbonate (3 mg) at RT. The reaction mixture was stirred for 1 h. Then the reaction mixture was loaded to a column and eluted with DCM 1% and 4% MeOH : DCM to afford compound 22 (15 mg, 54%). MS (ESI): 556 (MH⁺).

The compounds below were synthesized in a similar manner to that described in Examples 1-3.

| Cmpd # | Structure | Name | [M+H]⁺ |
|---|---|---|---|
| 23 | 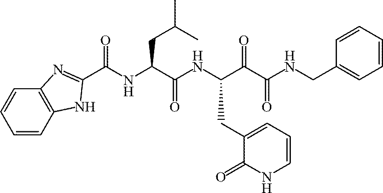 [SMILES: CC(C)C[C@H](NC(=O)c1nc2ccccc2[nH]1)C(=O)N[C@@H](Cc1ccc[nH]c1=O)C(=O)C(=O)NCc1ccccc1] | N-((S)-1-(((S)-4-(benzylamino)-3,4-dioxo-1-(2-oxo-1,2-dihydropyridin-3-yl)butan-2-yl)amino)-4-methyl-1-oxopentan-2-yl)-1H-benzo[d]imidazole-2-carboxamide | 557 |
| 24 | 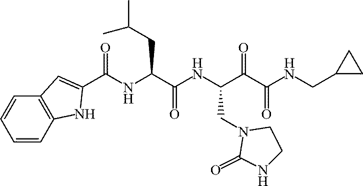 [SMILES: CC(C)C[C@H](NC(=O)c1cc2ccccc2[nH]1)C(=O)N[C@@H](CN1CCNC1=O)C(=O)C(=O)NCC1CC1] | N-((S)-1-(((S)-4-((cyclopropylmethyl)amino)-3,4-dioxo-1-(2-oxoimidazolidin-1-yl)butan-2-yl)amino)-4-methyl-1-oxopentan-2-yl)-1H-indole-2-carboxamide | 511 |
| 25 | 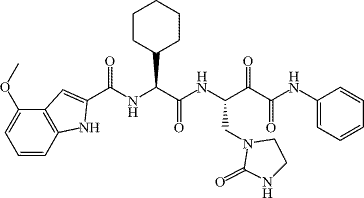 [SMILES: COc1cccc2[nH]c(C(=O)N[C@H](C(=O)N[C@@H](CN3CCNC3=O)C(=O)C(=O)Nc3ccccc3)C3CCCCC3)cc12] | N-((S)-1-cyclohexyl-2-(((S)-3,4-dioxo-1-(2-oxoimidazolidin-1-yl)-4-(phenylamino)butan-2-yl)amino)-2-oxoethyl)-4-methoxy-1H-indole-2-carboxamide | 589 |
| 26 | 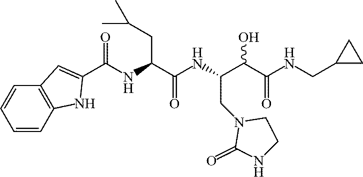 [SMILES: CC(C)C[C@H](NC(=O)c1cc2ccccc2[nH]1)C(=O)N[C@@H](CN1CCNC1=O)C(O)C(=O)NCC1CC1] | N-((2S)-1-(((2S)-4-((cyclopropylmethyl)amino)-3-hydroxy-4-oxo-1-(2-oxoimidazolidin-1-yl)butan-2-yl)amino)-4-methyl-1-oxopentan-2-yl)-1H-indole-2-carboxamide | 513 |
| 27 | 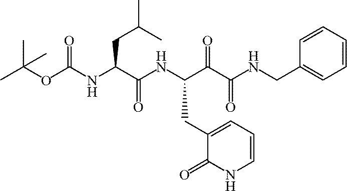 [SMILES: CC(C)C[C@H](NC(=O)OC(C)(C)C)C(=O)N[C@@H](Cc1ccc[nH]c1=O)C(=O)C(=O)NCc1ccccc1] | tert-butyl ((S)-1-(((S)-4-(benzylamino)-3,4-dioxo-1-(2-oxo-1,2-dihydropyridin-3-yl)butan-2-yl)amino)-4-methyl-1-oxopentan-2-yl)carbamate | 513 |
| 30 | 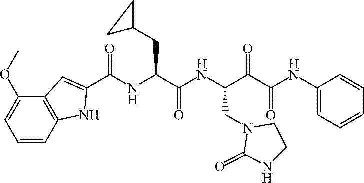 [SMILES: COc1cccc2[nH]c(C(=O)N[C@@H](CC3CC3)C(=O)N[C@@H](CN3CCNC3=O)C(=O)C(=O)Nc3ccccc3)cc12] | N-((S)-3-cyclopropyl-1-(((S)-3,4-dioxo-1-(2-oxoimidazolidin-1-yl)-4-(phenylamino)butan-2-yl)amino)-1-oxopropan-2-yl)-4-methoxy-1H-indole-2-carboxamide | 561 |
| 31 | 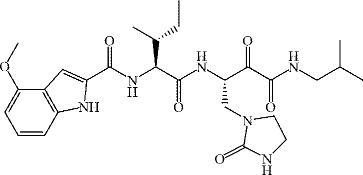 [SMILES: CC[C@@H](C)[C@H](NC(=O)c1cc2c(OC)cccc2[nH]1)C(=O)N[C@@H](CN1CCNC1=O)C(=O)C(=O)NCC(C)C] | N-((2S,3R)-1-(((S)-4-(isobutylamino)-3,4-dioxo-1-(2-oxoimidazolidin-1-yl)butan-2-yl)amino)-3-methyl-1-oxopentan-2-yl)-4-methoxy-1H-indole-2-carboxamide | 543 |
| 32 | 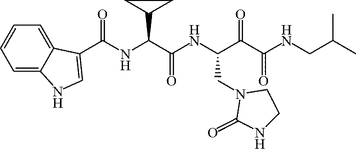 [SMILES: CC(C)CNC(=O)C(=O)[C@H](CN1CCNC1=O)NC(=O)[C@@H](NC(=O)c1c[nH]c2ccccc12)C1CC1] | N-((S)-1-cyclopropyl-2-(((S)-4-(isobutylamino)-3,4-dioxo-1-(2-oxoimidazolidin-1-yl)butan-2-yl)amino)-2-oxoethyl)-1H-indole-3-carboxamide | 497 |
| 33 | 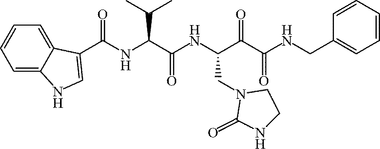 [SMILES: CC(C)[C@H](NC(=O)c1c[nH]c2ccccc12)C(=O)N[C@@H](CN1CCNC1=O)C(=O)C(=O)NCc1ccccc1] | N-((S)-1-(((S)-4-(benzylamino)-3,4-dioxo-1-(2-oxoimidazolidin-1-yl)butan-2-yl)amino)-3-methyl-1-oxobutan-2-yl)-1H-indole-3-carboxamide | 533 |
| 34 | 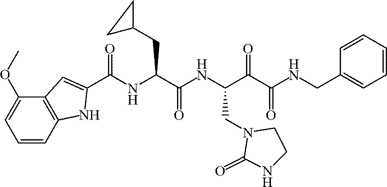 [SMILES: COc1cccc2[nH]c(C(=O)N[C@@H](CC3CC3)C(=O)N[C@@H](CN3CCNC3=O)C(=O)C(=O)NCc3ccccc3)cc12] | N-((S)-1-(((S)-4-(benzylamino)-3,4-dioxo-1-(2-oxoimidazolidin-1-yl)butan-2-yl)amino)-3-cyclopropyl-1-oxopropan-2-yl)-4-methoxy-1H-indole-2-carboxamide | 575 |
| 35 | 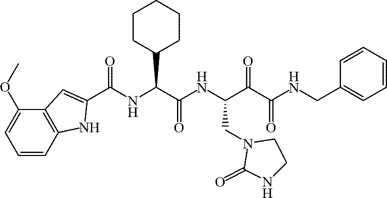 [SMILES: COc1cccc2[nH]c(C(=O)N[C@H](C(=O)N[C@@H](CN3CCNC3=O)C(=O)C(=O)NCc3ccccc3)C3CCCCC3)cc12] | N-((S)-2-(((S)-4-(benzylamino)-3,4-dioxo-1-(2-oxoimidazolidin-1-yl)butan-2-yl)amino)-1-cyclohexyl-2-oxoethyl)-4-methoxy-1H-indole-2-carboxamide | 603 |
| 36 | 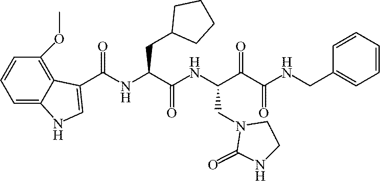 [SMILES: COc1cccc2[nH]cc(C(=O)N[C@@H](CC3CCCC3)C(=O)N[C@@H](CN3CCNC3=O)C(=O)C(=O)NCc3ccccc3)c12] | N-((S)-1-(((S)-4-(benzylamino)-3,4-dioxo-1-(2-oxoimidazolidin-1-yl)butan-2-yl)amino)-3-cyclopentyl-1-oxopropan-2-yl)-4-methoxy-1H-indole-3-carboxamide | 603 |
| 37 | 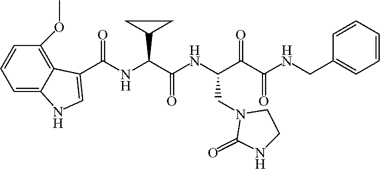 [SMILES: COc1cccc2[nH]cc(C(=O)N[C@H](C(=O)N[C@@H](CN3CCNC3=O)C(=O)C(=O)NCc3ccccc3)C3CC3)c12] | N-((S)-2-(((S)-4-(benzylamino)-3,4-dioxo-1-(2-oxoimidazolidin-1-yl)butan-2-yl)amino)-1-cyclopropyl-2-oxoethyl)-4-methoxy-1H-indole-3-carboxamide | 561 |
| 38 | 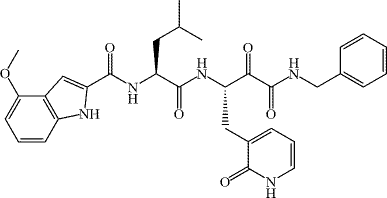 [SMILES: COc1cccc2[nH]c(C(=O)N[C@@H](CC(C)C)C(=O)N[C@@H](Cc3ccc[nH]c3=O)C(=O)C(=O)NCc3ccccc3)cc12] | N-((S)-1-(((S)-4-(benzylamino)-3,4-dioxo-1-(2-oxo-1,2-dihydropyridin-3-yl)butan-2-yl)amino)-4-methyl-1-oxopentan-2-yl)-4-methoxy-1H-indole-2-carboxamide | 586 |
| 39 | 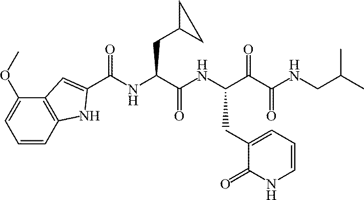 [SMILES: COc1cccc2[nH]c(C(=O)N[C@@H](CC3CC3)C(=O)N[C@@H](Cc3ccc[nH]c3=O)C(=O)C(=O)NCC(C)C)cc12] | N-((S)-3-cyclopropyl-1-(((S)-4-(isobutylamino)-3,4-dioxo-1-(2-oxo-1,2-dihydropyridin-3-yl)butan-2-yl)amino)-1-oxopropan-2-yl)-4-methoxy-1H-indole-2-carboxamide | 550 |
| 40 | 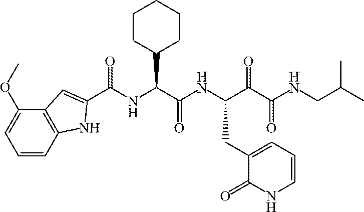 [SMILES: COc1cccc2[nH]c(C(=O)N[C@H](C(=O)N[C@@H](Cc3ccc[nH]c3=O)C(=O)C(=O)NCC(C)C)C3CCCCC3)cc12] | N-((S)-1-cyclohexyl-2-(((S)-4-(isobutylamino)-3,4-dioxo-1-(2-oxo-1,2-dihydropyridin-3-yl)butan-2-yl)amino)-2-oxoethyl)-4-methoxy-1H-indole-2-carboxamide | 578 |

### Example 4: Synthesis of N-((S)-1-(((S)-4-(Benzylamino)-3,4-Dioxo-1-(2-Oxo-1,2-Dihydropyridin-3-yl)Butan-2-yl)Amino)-4-Methyl-1-Oxopentan-2-yl)-1H-Imidazole-2-Carboxamide (28) and N-((S)-1-(((S)-4-(Benzylamino)-3,4-Dioxo-1-(2-Oxo-1,2-Dihydropyridin-3-yl)butan-2-Yl)Amino)-4-Methyl-1-Oxopentan-2-yl)-1H-Imidazole-5-Carboxamide (29)

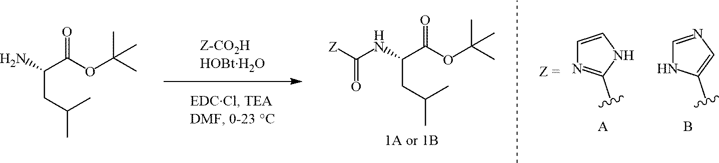

To a solution of leucine tert-butyl ester (10 mmol) in DMF (150 mL) at 23° C. was added carboxylic acid A or B (12 mmol), HOBt-H₂O (12 mmol) and EDC-HCl (12 mmol). The resulting solution was cooled to 0° C., and triethylamine (12 mmol) was added dropwise. The solution was allowed to warm to ambient temperature and stirred for 2 hours, after which time the solvent was removed in vacuo, and the resulting residue was dissolved in EtOAc (200 mL). The organic layer was washed with 5% citric acid (2 × 200 mL), 5% NaHCO₃ (2 × 200 mL), and brine (200 mL). The organic layers were dried over Na₂SO₄, filtered, and concentrated in vacuo. The crude residue was purified by flash column chromatography to afford the product.

### Synthesis of Compounds 2A and 2B

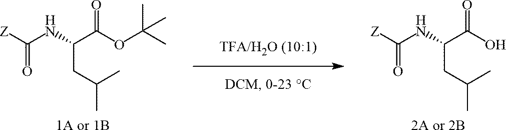

To a solution of tert-butyl ester 1A or 1B (8 mmol) in dichloromethane (8 mL) at 0° C. was added TFA:H₂O (10:1, 12 mL). The resulting mixture was stirred for 5 minutes, and then was warmed to room temperature and stirred for a further 1 h. The solvent was then removed in vacuo to afford the product, which was used in the next step without further purification.

### Synthesis of Compounds 3A and 3B

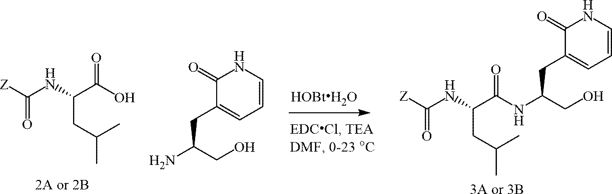

To a solution of (S)-3-(2-amino-3-hydroxypropyl)pyridin-2(1H)-one (5 mmol) in DMF (75 mL) at 23° C. was added carboxylic acid 2A or 2B (6 mmol), HOBt-H₂O (6 mmol) and EDC-HCl (6 mmol). The resulting solution was cooled to 0° C., and triethylamine (6 mmol) was added dropwise. The solution was allowed to warm to ambient temperature and stirred for 2 hours, after which time the solvent was removed in vacuo, and the resulting residue was dissolved in EtOAc (100 mL). The organic layer was washed with 5% citric acid (2 × 100 mL), 5% NaHCO₃ (2 × 100 mL), and brine (100 mL). The organic layers were dried over Na₂SO₄, filtered, and concentrated in vacuo. The crude residue was purified by flash column chromatography to afford the product.

Compound 3A: MS (ESI): 376 (MH⁺). Compound 3B: MS (ESI): 376 (MH⁺).

### Synthesis of Compounds 4A and 4B

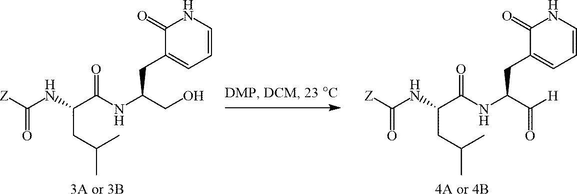

To a solution of alcohol 3A or 3B (4 mmol) in dichloromethane (20 mL) was added Dess-Martin periodinane (5.2 mmol). The resulting mixture was stirred for 2 h at room temperature, and then quenched with a saturated solution aqueous solution of sodium thiosulfate (20 mL). The mixture was stirred for 10 minutes, combined with aqueous saturated sodium bicarbonate (30 mL), stirred for a further 15 minutes, and extracted with ethyl acetate (3 × 50 mL). The combined organic layers were washed with aqueous saturated sodium bicarbonate (20 mL) and brine (20 mL), dried over MgSO₄, and concentrated in vacuo to afford the product.

### Synthesis of Compounds 5A and 5B

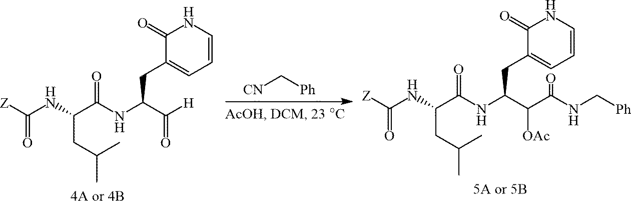

To a solution of aldehyde 4A or 4B (3 mmol) in dichloromethane (15 mL) was added benzyl isocyanide (5.4 mmol) and acetic acid (5.4 mmol). The mixture was stirred for 12 hours, concentrated in vacuo, and purified via flash column chromatography to afford the product. Compound 5A: MS (ESI): 551 (MH⁺). Compound 5B: MS (ESI): 551 (MH⁺).

### Synthesis of Compounds 6A and 6B

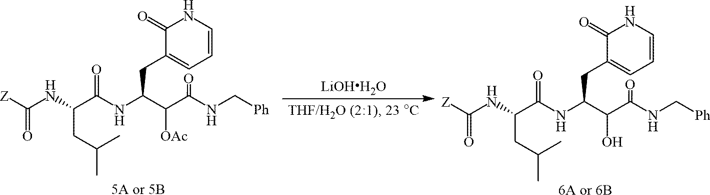

To a solution of acetate 5A or 5B (2 mmol) in THF:H₂O (2:1, 15 mL) was added LiOH-H₂O (4 mmol). The solution was stirred for 1 h, diluted with aqueous saturated sodium bicarbonate solution (30 mL) and extracted with ethyl acetate (3 × 60 mL). The combined organic layers were dried over MgSO₄, filtered, and concentrated in vacuo to afford the product, which was used in the next step without further purification. Compound 6A: MS (ESI): 509 (MH⁺). Compound 6B: MS (ESI): 509 (MH⁺).

### Synthesis of Compounds 28 and 29

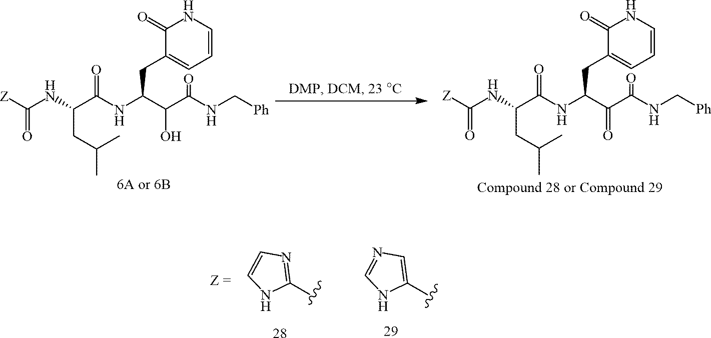

To a solution of hydroxyamide 6A or 6B (1 mmol) in dichloromethane (15 mL) was added Dess-Martin periodinane (1.3 mmol). The resulting mixture was stirred for 1 h at room temperature, and then quenched with a saturated solution aqueous solution of sodium thiosulfate (20 mL). The mixture was stirred for 10 minutes, combined with aqueous saturated sodium bicarbonate (30 mL), stirred for a further 15 minutes, and extracted with ethyl acetate (3 × 50 mL). The combined organic layers were washed with aqueous saturated sodium bicarbonate (20 mL) and brine (20 mL), dried over MgSO₄, and purified via flash column chromatography to afford the respective products, Compound 28 and Compound 29. Compound 28: MS (ESI): 507 (MH⁺). Compound 29: MS (ESI): 507 (MH⁺).

### Example 5: In Silico Screening of Alpha-Ketoamide Inhibitors of SARS-COV2 Mpro

A series of structures based on a scaffold according to Formula (A) were enumerated and then subjected to a series of in silico screening procedures via the DataWarrior software package to disqualify candidates on the basis of postulated mutagenicity, tumorigenicity, irritant effects, and adverse reproductive effects (Thomas Sander, Joel Freyss, Modest von Korff, Christian Rufener. DataWarrior: An Open-Source Program for Chemistry Aware Data Visualization and Analysis. J. Chem. Inf. Model. 2015, 55, 460-473).

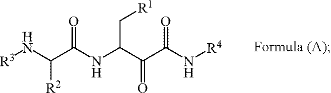

wherein:
R¹ is a heteroaromatic 5, 6 or 7 membered ring system providing a hydrogen bond acceptor and a hydrogen bond donor;
R² is C₂-C₇ alkyl, C₂-C₇ cycloalkyl or, aryl substituted C₂-C₇ alkyl (e.g. ethyl, propyl, 2-methyl-ethyl, n-butyl, iso-butyl, methyl-cyclopropyl, methyl-cyclobutyl, methylcyclopentyl, methyl-cyclohexyl, benzyl, and fluorosubstituted benzyl);
R³ is a nitrogen-containing heteroaryl or heterocycloalkyl, or acyl substituted with a nitrogen-containing heteroaryl or heterocycloalkyl; and
R⁴ is alkyl, cycloalkyl, aryl, or aralkyl.

The resulting library of 4852 structures was then docked into the catalytic pocket of SARS-COV2 Mpro (PDB ID: 642F, Science, 2020, 368, 409-412) using the MOLOC software package. A selection of 1326 structures were identified that exhibited a greater calculated binding affinity (arbitrary molecular dynamic force field < -1300 kJ/mol) than reference Compound 1Z (-1235.11 kJ/mol).

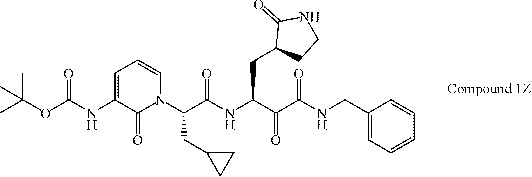 [SMILES: CC(C)(C)OC(=O)Nc1cccn([C@@H](CC2CC2)C(=O)N[C@@H](C[C@@H]2CCNC2=O)C(=O)C(=O)NCc2ccccc2)c1=O]

A further sub-selection of compounds was made based on the following calculated properties: clogP, clogS, polar surface area, and drug-likeness score.

### ClogP

The calculated logP (clogP) is the logarithm of its partition coefficient between n-octanol and water log(coctanol/cwater). clogP is a well-established measure of a compound’s hydrophilicity. Low hydrophilicities and therefore high logP values cause poor absorption or permeation. It has been shown for compounds to have a reasonable probability of being well absorpt if their clogP is larger than 0 and smaller than 4.0 (Tetko et al. “Calculation of molecular lipophilicity: State-of-the-art and comparison of log P methods on more than 96,000 compounds”, J. Pharm. Sci. 2009, 98 (3), 861-93).

### ClogS

The aqueous solubility of a compound significantly affects its absorption and distribution characteristics. A low solubility goes along with a bad absorption and therefore the general aim is to avoid poorly soluble compounds - more than 80% of the drugs on the market have a calculated logS (clogS) greater than -4. The clogS is the logarithm of the solubility in mol/liter at pH 7.5 and 25 C.

### Polar Surface Area

Molecules with a polar surface area of greater than 140 angstroms squared tend to be poor at permeating cell membranes (Pajouhesh H, Lenz GR “Medicinal Chemical Properties of Successful Central Nervous System Drugs”, NeuroRx. 2 (4): 541-553.).

### Drug-Likeness

The calculation of drug-likeness is based on a list of about 5300 distinct substructure fragments with associated drug likeness scores. It is calculated using OSIRIS (Sander et al. “OSIRIS, an Entirely in-House Developed Drug Discovery Informatics System”, Journal of Chemical Information and Modeling 2009, 49(2):232-46). The majority of drugs has a drug likeness score of greater than 0.

Based on the calculations above, a further sub-selection of compounds (Table 1) that exhibited a calculated clogP between 0 and 2, a polar surface area of less than 165, and drug-likeness score of greater than 2 were chosen for synthesis and biological screening.

Compounds of Table 1 are prepared using the procedures described in Examples 1-4.

TABLE 1

| Compound | Structure | cLogP | cLogS | Polar Surface Area | Drug-likeness |
|---|---|---|---|---|---|
| A1 | 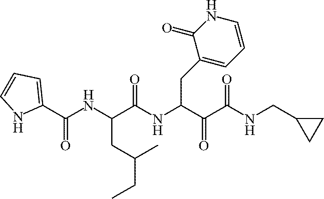 [SMILES: CCC(C)CC(NC(=O)c1ccc[nH]1)C(=O)NC(Cc1ccc[nH]c1=O)C(=O)C(=O)NCC1CC1] | 0.021 | -3.18 | 149.26 | 3.1241 |
| A2 | 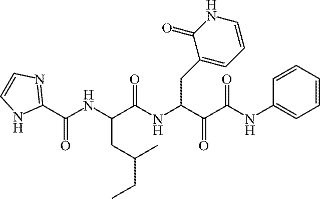 [SMILES: CCC(C)CC(NC(=O)c1ncc[nH]1)C(=O)NC(Cc1ccc[nH]c1=O)C(=O)C(=O)Nc1ccccc1] | 0.2785 | -2.922 | 162.15 | 2.1812 |
| A3 | 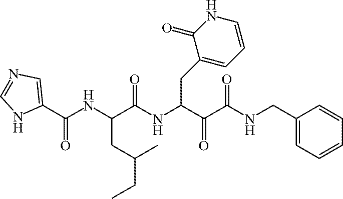 [SMILES: CCC(C)CC(NC(=O)c1cnc[nH]1)C(=O)NC(Cc1ccc[nH]c1=O)C(=O)C(=O)NCc1ccccc1] | 0.1524 | -3.478 | 162.15 | 3.3894 |
| A4 | 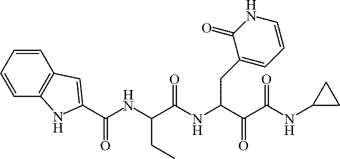 [SMILES: CCC(NC(=O)c1cc2ccccc2[nH]1)C(=O)NC(Cc1ccc[nH]c1=O)C(=O)C(=O)NC1CC1] | 0.0161 | -3.929 | 149.26 | 2.9369 |
| A5 | 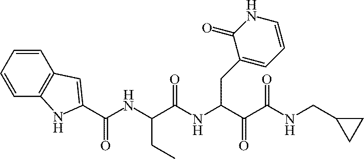 [SMILES: CCC(NC(=O)c1cc2ccccc2[nH]1)C(=O)NC(Cc1ccc[nH]c1=O)C(=O)C(=O)NCC1CC1] | 0.2108 | -3.981 | 149.26 | 4.1823 |
| A6 | 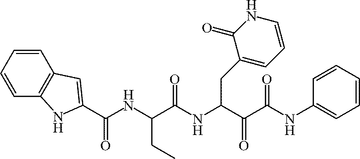 [SMILES: CCC(NC(=O)c1cc2ccccc2[nH]1)C(=O)NC(Cc1ccc[nH]c1=O)C(=O)C(=O)Nc1ccccc1] | 1.1951 | -4.672 | 149.26 | 2.7733 |
| A7 | 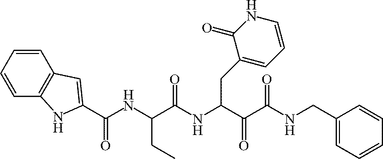 [SMILES: CCC(NC(=O)c1cc2ccccc2[nH]1)C(=O)NC(Cc1ccc[nH]c1=O)C(=O)C(=O)NCc1ccccc1] | 0.9045 | -4.488 | 149.26 | 3.0998 |
| A8 | 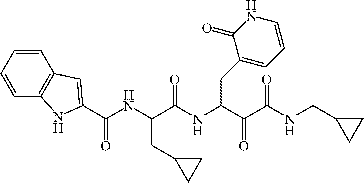 [SMILES: O=C(NCC1CC1)C(=O)C(Cc1ccc[nH]c1=O)NC(=O)C(CC1CC1)NC(=O)c1cc2ccccc2[nH]1] | 0.5289 | -4.497 | 149.26 | 2.8389 |
| A9 | 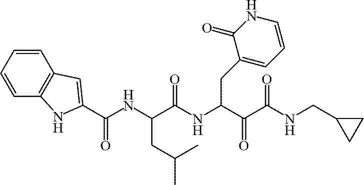 [SMILES: CC(C)CC(NC(=O)c1cc2ccccc2[nH]1)C(=O)NC(Cc1ccc[nH]c1=O)C(=O)C(=O)NCC1CC1] | 0.8835 | -4.411 | 149.26 | 2.8894 |
| A10 | 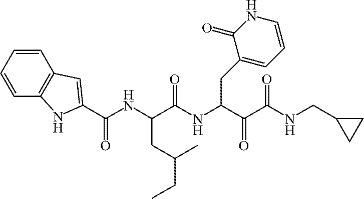 [SMILES: CCC(C)CC(NC(=O)c1cc2ccccc2[nH]1)C(=O)NC(Cc1ccc[nH]c1=O)C(=O)C(=O)NCC1CC1] | 1.3379 | -4.681 | 149.26 | 3.1241 |
| A11 | 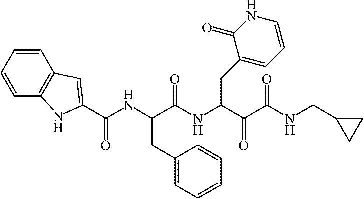 [SMILES: O=C(NCC1CC1)C(=O)C(Cc1ccc[nH]c1=O)NC(=O)C(Cc1ccccc1)NC(=O)c1cc2ccccc2[nH]1] | 1.1983 | -4.846 | 149.26 | 2.8389 |
| A12 | 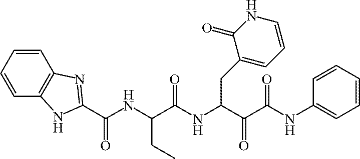 [SMILES: CCC(NC(=O)c1nc2ccccc2[nH]1)C(=O)NC(Cc1ccc[nH]c1=O)C(=O)C(=O)Nc1ccccc1] | 0.5908 | -3.618 | 162.15 | 3.2596 |
| A13 | 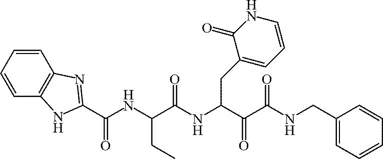 [SMILES: CCC(NC(=O)c1nc2ccccc2[nH]1)C(=O)NC(Cc1ccc[nH]c1=O)C(=O)C(=O)NCc1ccccc1] | 0.3002 | -3.434 | 162.15 | 3.5819 |
| A14 | 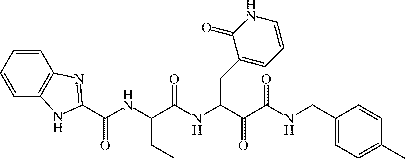 [SMILES: CCC(NC(=O)c1nc2ccccc2[nH]1)C(=O)NC(Cc1ccc[nH]c1=O)C(=O)C(=O)NCc1ccc(C)cc1] | 0.401 | -3.748 | 162.15 | 2.2419 |
| A15 | 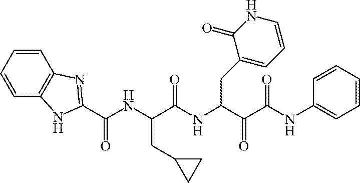 [SMILES: O=C(Nc1ccccc1)C(=O)C(Cc1ccc[nH]c1=O)NC(=O)C(CC1CC1)NC(=O)c1nc2ccccc2[nH]1] | 0.9089 | -4.134 | 162.15 | 2.2464 |
| A16 | 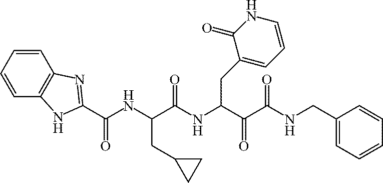 [SMILES: O=C(NCc1ccccc1)C(=O)C(Cc1ccc[nH]c1=O)NC(=O)C(CC1CC1)NC(=O)c1nc2ccccc2[nH]1] | 0.6183 | -3.95 | 162.15 | 2.5872 |
| A17 | 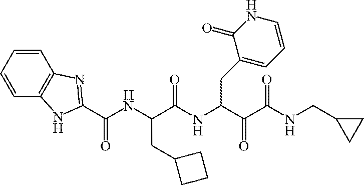 [SMILES: O=C(NCC1CC1)C(=O)C(Cc1ccc[nH]c1=O)NC(=O)C(CC1CCC1)NC(=O)c1nc2ccccc2[nH]1] | 0.2666 | -3.713 | 162.15 | 2.4318 |
| A18 | 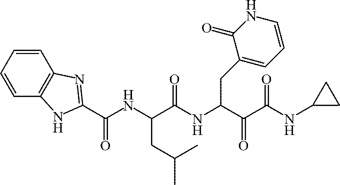 [SMILES: CC(C)CC(NC(=O)c1nc2ccccc2[nH]1)C(=O)NC(Cc1ccc[nH]c1=O)C(=O)C(=O)NC1CC1] | 0.0845 | -3.305 | 162.15 | 2.1052 |
| A19 | 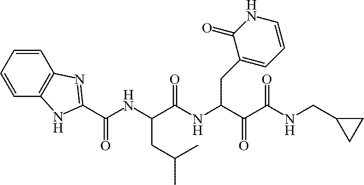 [SMILES: CC(C)CC(NC(=O)c1nc2ccccc2[nH]1)C(=O)NC(Cc1ccc[nH]c1=O)C(=O)C(=O)NCC1CC1] | 0.2792 | -3.357 | 162.15 | 3.3913 |
| A21 | 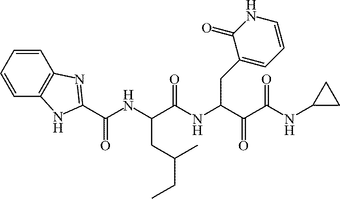 [SMILES: CCC(C)CC(NC(=O)c1nc2ccccc2[nH]1)C(=O)NC(Cc1ccc[nH]c1=O)C(=O)C(=O)NC1CC1] | 0.5389 | -3.575 | 162.15 | 2.3523 |
| A22 | 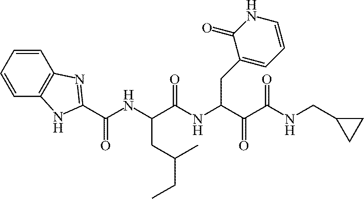 [SMILES: CCC(C)CC(NC(=O)c1nc2ccccc2[nH]1)C(=O)NC(Cc1ccc[nH]c1=O)C(=O)C(=O)NCC1CC1] | 0.7336 | -3.627 | 162.15 | 3.6219 |
| A23 | 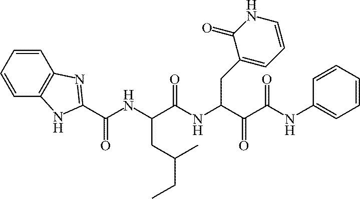 [SMILES: CCC(C)CC(NC(=O)c1nc2ccccc2[nH]1)C(=O)NC(Cc1ccc[nH]c1=O)C(=O)C(=O)Nc1ccccc1] | 1.7179 | -4.318 | 162.15 | 2.1812 |
| A24 | 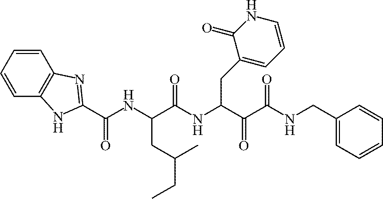 [SMILES: CCC(C)CC(NC(=O)c1nc2ccccc2[nH]1)C(=O)NC(Cc1ccc[nH]c1=O)C(=O)C(=O)NCc1ccccc1] | 1.4273 | -4.134 | 162.15 | 2.5402 |
| A25 | 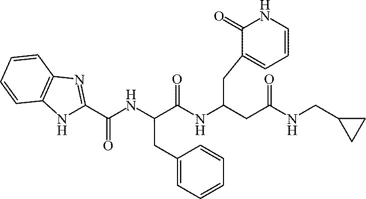 [SMILES: O=C(CC(Cc1ccc[nH]c1=O)NC(=O)C(Cc1ccccc1)NC(=O)c1nc2ccccc2[nH]1)NCC1CC1] | 0.594 | -3.792 | 162.15 | 3.3225 |
| A26 | 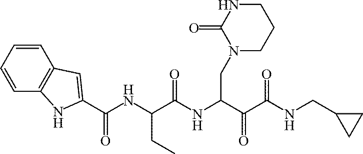 [SMILES: CCC(NC(=O)c1cc2ccccc2[nH]1)C(=O)NC(CN1CCCNC1=O)C(=O)C(=O)NCC1CC1] | 0.1325 | -3.468 | 152.5 | 6.072 |
| A27 | 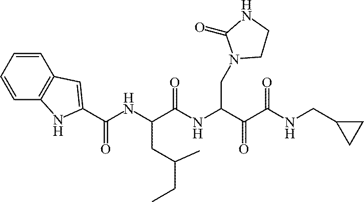 [SMILES: CCC(C)CC(NC(=O)c1cc2ccccc2[nH]1)C(=O)NC(CN1CCNC1=O)C(=O)C(=O)NCC1CC1] | 0.9176 | -3.898 | 152.5 | 5.7074 |
| A28 | 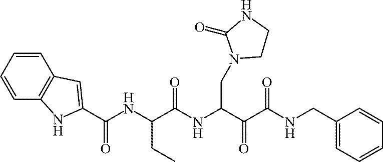 [SMILES: CCC(NC(=O)c1cc2ccccc2[nH]1)C(=O)NC(CN1CCNC1=O)C(=O)C(=O)NCc1ccccc1] | 0.4842 | -3.705 | 152.5 | 5.5598 |
| A29 | 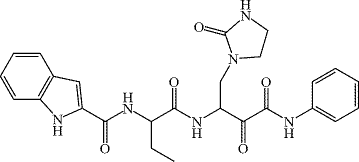 [SMILES: CCC(NC(=O)c1cc2ccccc2[nH]1)C(=O)NC(CN1CCNC1=O)C(=O)C(=O)Nc1ccccc1] | 0.7748 | -3.889 | 152.5 | 5.5598 |
| 1Z | 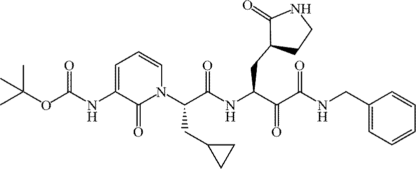 [SMILES: CC(C)(C)OC(=O)Nc1cccn([C@@H](CC2CC2)C(=O)N[C@@H](C[C@@H]2CCNC2=O)C(=O)C(=O)NCc2ccccc2)c1=O] | 0.8091 | -4.652 | 163.01 | -52.896 |

### Biological Assays

### Example 6: Mpro Enzyme Activity Assay for Inhibitory Compounds

Compounds were tested for their capability to inhibit the SARS-CoV-2 main protease M^{pro} by using a biochemical FRET-based M^{pro} enzyme activity assay. Recombinant M^{pro} protein (Ser1 - Gln306; with proven proteolytic activity) was purchased from Biosynth Carbosynth (Staad, Switzerland). An EDANS- and Dabcyl-labeled peptide -[Dabcyl]KTSAVLQ↓SGFRKM[Glu(EDANS)]-amide (M^{pro} cleavage site indicated by the arrow) -was purchased from Life Technologies GmbH (Darmstadt, Germany), and served as substrate peptide for M^{pro} proteolytic cleavage allowing fluorescence resonance energy transfer (FRET) read-out. Due to the M^{pro}-mediated cleavage of the substrate peptide, the EDANS fluorescence (λexc. = 336 nm; λem. = 490 nm) becomes dequenched (from disappearing Dabcyl) and increases with increasing M^{pro} activity. The assay buffer was 20 mM Tris buffer supplemented with 100 mM NaCl, and 1 mM EDTA, adjusted to pH 7.3 with 1N HCl. The test compounds were diluted from 20 mM stocks in DMSO; the stock of the substrate peptide was 250 µM in aqua bidest. The catalytic activity of the recombinant M^{pro} enzyme was 20 U/mg. It was checked in advance that neither the assay buffer nor the M^{pro} protein by itself emit fluorescence at 490 nm under 336 nm excitation. The basal emission of the uncleaved substrate peptide was subtracted from all results by baseline correction. The enzyme assay was carried out in black U-form half-area 96-wells. Each assay sample was finally composed of 0.4 µL substrate peptide stock (3X ad 20 µL assay buffer to yield finally 2 µM; 100 pmol), 0.1 µL Mpro enzyme (20 mU in assay buffer ad 20 µL) and 20 µL of 3X (in assay buffer) test compound dilution, resulting in a final sample volume of 60 µL. The final test compound concentrations were: 10 µM for compound fast-screening, and 0 - 200 µM for IC₅₀ determinations. Initially, Mpro enzyme and test compound was added and mixed in 96 -well and pre-incubated for 30 min in the dark with 200 rpm swiveling at room temperature. Subsequently, the reaction was started by addition of the substrate peptide, and followed by a fluorescence kinetic (λexc. = 336 nm / λem. = 500 nm / CutOff = 435 nm; 30 min with 2 min increment by using a SpectraMax M5 multiwell plate reader (Molecular Devices, San Jose, CA, USA). Pure assay buffer served as blank control, samples containing just Mpro enzyme and substrate peptide without test compound were measured as positive control, and samples containing heat-inactivated (10 min at 60° C.) Mpro enzyme and substrate peptide as negative control. Each data point was tested in technical duplicates and ≥ 3 biological replicates. Dose-response curves were analyzed by using GraphPad Prism 8.0 software resulting in IC₅₀ values (see Table 2, “Mpro enzyme inhibition”).

### Example 7: In Vitro Cell Viability Assay for Evaluation of Compound Side Effects

### Cell Culture

The human colon adenocarcinoma cell line CACO-2 was purchased from the German Collection of Microorganisms and Cell Culture GmbH (DSMZ, Braunschweig, Germany). The human lung adenocarcinoma cell line Calu-3 was purchased form ATCC (Manassas, USA). CACO-2 cells were cultured in MEM medium supplemented with 10% heat-inactivated FBS and 1X non-essential amino acids (NEAA) and Calu-3 cells in DMEM supplemented with 10% heat-inactivated FCS, 1% antibiotics (Pen/Strep solution) and 1% Non-Essential Amino Acids, respectively, in a humidified incubator at 37° C. with 5% CO2. All media and supplements were purchased from Capricorn Scientific GmbH (Ebsdorfergrund, Germany), all cell culture plastics from TPP (Trasadingen, Switzerland) and Greiner Bio-One International GmbH (Kremsmünster, Austria), respectively.

### Cell Viability Assay (Compound Side Effects)

Since cytotoxic effects of the test compounds towards the human cells to be rescued from viral infection are undesired, test items were initially checked for potential cytotoxic effects towards the cell lines under investigation by using a fluorometric Resazurin-based in vitro cell viability assay. In brief, CACO-2 (colon adenocarcinoma) cells were seeded with low densities into 96-well plates (5,000 cells per well), and were allowed to adhere for 24 h. Subsequently, the test compounds - dissolved to the concentration range 0.39 - 100 µM (factor 2 dilutions; max. 0.5% DMSO finally) in cell line-specific medium - were added to the cells and incubated for 72 h. Subsequently, medium was replaced by 50 uM resazurin in RPMI 1640, and the cells were incubated for 2 h. Finally, the conversion of resazurin to Resorufin by viable, metabolically active cells was measured using a SpectraMax M5 multiwell plate reader (Molecular Devices, San Jose, CA, USA) with 540 nm excitation and 590 nm emission filter setting. Dose-response curves were analyzed by using GraphPad Prism 8.0 software resulting in IC₅₀ values. DMSO and resazurin were purchased from Sigma-Aldrich (St. Louis, USA). Furthermore, in conjunction with the SARS-CoV-2 antiviral assays (PRA and VYRA), the cytotoxicity of the compounds in the antiviral Calu-3 assay setting was additionally evaluated by AlamarBlue reduction assay. Calu-3 cells were seeded in 96-well plates in an initial density of 2 × 10⁴ cells per well. The cells were incubated with increasing concentrations of the compounds for 48 h at 37° C. and 5% CO₂. AlamarBlue HS Cell Viability Reagent (Thermo Fisher Scientific) was added to the cells, which were further incubated for 4 h at 37° C. The optical density was measured at 570 nm and 600 nm and the percentage of viable cells was calculated according to manufacturer’s instructions. Compounds were tested in the range of 0.0001 - 100 µM in 1:3 intervals, and results were expressed as the percentage of viable cells in treated samples, taking as reference molarity-matched DMSO controls. All tested compounds were shown to be non-toxic for the human cells under investigation beyond concentrations of 100 µM, even after 72 h treatment (see Table 2, “Cytotoxicity”).

TABLE 2

| Compound | Mpro enzyme inhibition IC₅₀ [µM] | Cytotoxicity CC₅₀ [µM] |
|---|---|---|
| 10 | ++ | > 100 |
| 12 | ++ | > 100 |
| 22 | ++ | > 100 |
| 23 | ++ | > 100 |
| 24 | + | > 100 |
| 25 | ++ | > 100 |
| 26 | ++ | > 100 |
| 27 | + | > 100 |

where ‘++’ means IC₅₀ ≤ 2.0 µM; where ‘+’ means 2.0 µM < IC₅₀ ≤ 20 µM.

### Example 8: In Vitro Cell Viability Assay for Screening of Antiviral Compounds

### Virus Culture

SARS-CoV-2 virus is isolated from patients in authorized German hospitals, e.g. University hospital Frankfurt/Main. SARS-CoV-2 is propagated in human CACO-2 cells and stored as stocks at -80° C. Virus titers (TCIP50/mL) are determined in dense but still subconfluent CACO-2 in 96-well.

### Cell Viability Assay (Screening)

Test compounds are screened for anti-SARS-CoV-2 activity by using virus-infected CACO-2 cells. In brief, test compounds are diluted to appropriate concentrations in MEM supplemented with 1% FBS (reduced FBS content) - 10 µM (0.1% DMSO finally) for initial compound fast-screening and 0.02 - 50 µM (max. 0.5% DMSO finally) for IC50 analyses, respectively - and added to dense but still subconfluent CACO-2 cells in 96-well plates. Subsequently, the cells are infected immediately with SARS-CoV-2 (MOI = 0.01). Non-treated cells - with and without virus infection - serve as controls and contain the corresponding max. final DMSO concentration as well. Each data point is tested in triplicates. After 48 h treatment, the cells are either fixed and inspected by high content imaging or stained with resazurin cell viability staining.

In both cases the viability/presence of intact CACO-2 cells served as measure to quantify the virus inhibition by the test compounds. In case of imaging quantification, the cells are fixed with 3% PFA in PBS, the 96-well plates are sealed and SARS-CoV-2 is inactivated by disinfection. The quan-tification is conducted by using an Operetta CLS (PerkinElmer) - in label-free mode just using the digital maximum phase contrast, and with cell nuclei staining using Hoechst 33258 (Sigma-Aldrich). For that purpose, cell images are acquired using a 10X objective, and 3×3 imaged field per 96-well are analyzed. In case of resazurin-based cell viability read-out, after 48 h cell treat-ment, medium is replaced by 50 µM resazurin in RPMI 1640, and the cells are incubated for 2 h. Subsequently, the conversion of resazurin to Resorufin by viable, metabolically active cells is measured using a Synergy 2 multiwell plate reader (BioTek, Bad Friedrichshall, Germany) with 540 nm excitation and 590 nm emission filter setting.

Results are normalized to the corresponding intra-plate non-treated controls, whereby without virus re-flected 100% cell viability or 0% viral cytotoxicity = 100% inhibition of viral toxicity; with virus re-flected 0% cell viability or 100% viral cytotoxicity = 0% inhibition of viral toxicity. Dose-response curves are analyzed by using GraphPad Prism resulting in IC50 values.

### Example 9: In Vitro Viral Load Assay for Screening of Antiviral Compounds

An RT-qPCR-based viral load assay is used to screen the test items for anti-SARS-CoV-2 activity. In brief, test compounds are diluted to appropriate concentrations in MEM supplemented with 1% FBS (reduced FBS content) - 10 µM (0.1% DMSO finally) for initial compound fast-screening and 0.02 - 50 µM (max. 0.5% DMSO finally) for IC50 analyses, respectively - and added to dense but still subconfluent CACO-2 cells in 96-well plates. Subsequently, the cells are infected immediately with SARS-CoV-2. Non-treated cells - with and without virus infection -serve as controls and contain the corresponding max. final DMSO concentration as well. Each data point is tested in triplicates. After 72 h treatment, cell supernatants are collected and centrifuged (2,000 rpm, 5 min) to remove cells and cell debris. Subsequently, viral RNAs are extracted from the cell supernatants by using a MagNA Pure 24 system (Roche, Mannheim, Germany). The RNA of SARS-CoV-2 is quantified using the TIB MOLBIOL LightMix Assay SARS-CoV-2 RdRP RT-qPCR assay kit with RNA Process Control PCR Kit (Roche), all according to the manufacturer’s guidelines. PCR amplification is conducted by using a LightCycler 480 II (Roche). ΔΔCt values are taken as measures for SARS-CoV-2 viral replication. Dose-response curves for compound-mediated inhibition of the viral replication are analyzed by using GraphPad Prism resulting in IC50 values.

The examples and embodiments described herein are for illustrative purposes only and in some embodiments, various modifications or changes are to be included within the purview of disclosure and scope of the appended claims.

What is claimed is:

1. A compound of Formula (I′), or a pharmaceutically acceptable salt or solvate thereof: 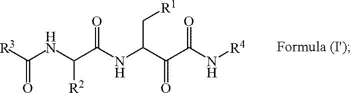
wherein:
R¹ is selected from 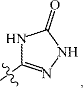 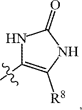 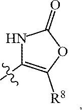 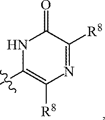 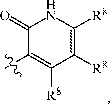 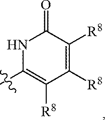 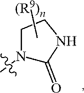 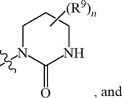 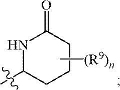
R² is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵,—C(O)N(R⁵)(R⁶), —S(O)₂R⁷, and —S(O)₂N(R⁵)(R⁶)—;
R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R⁴ is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵,—C(O)N(R⁵)(R⁶), —S(O)₂R⁷, and —S(O)₂N(R⁵)(R⁶)—;
each R⁵ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁶ is independently selected from hydrogen, C₁₋₆alkyl, and C₁₋₆haloalkyl; and
each R⁷ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁸ is independently selected from hydrogen, halogen, C₁₋₆alkyl, C₁₋₆haloalkyl, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and
each R⁹ is independently selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and
n is 0, 1, 2, 3, or 4.

2. A compound of Formula (I), or a pharmaceutically acceptable salt or solvate thereof: 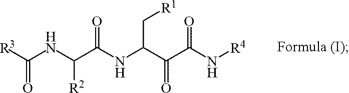
wherein:
R¹ is selected from 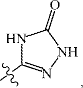 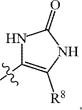 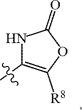 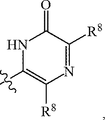 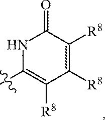 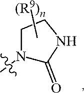 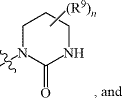 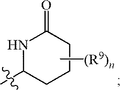
R² is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵,—C(O)N(R⁵)(R⁶), —S(O)₂R⁷, and —S(O)₂N(R⁵)(R⁶)—;
R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—;
R⁴ is selected from C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₂₋₆alkenyl, C₂₋₆alkynyl, C₃₋₆cycloalkyl, -C₁₋₆alkyl-C₃₋₆cycloalkyl, C₂₋₉heterocycloalkyl, -C₁₋₆alkyl-C₂₋₉heterocycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵,—C(O)N(R⁵)(R⁶), —S(O)₂R⁷, and —S(O)₂N(R⁵)(R⁶)—;
each R⁵ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally sub stituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁶ is independently selected from hydrogen, C₁₋₆alkyl, and C₁₋₆haloalkyl; and
each R⁷ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R⁸ is independently selected from hydrogen, halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and
each R⁹ is independently selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—; and
n is 0, 1, 2, 3, or 4.

3. The compound of claim 1 or claim 2, or a pharmaceutically acceptable salt or solvate thereof, wherein R⁴ is selected from C₁₋₆alkyl, C₃₋₆cycloalkyl, -C₁₋₆alkyl-C₃₋₆cycloalkyl, C₆₋₁₀aryl, -C₁₋₆alkyl-C₆₋₁₀aryl, C₁₋₉heteroaryl, and -C₁₋₆alkyl-C₁₋₉heteroaryl, wherein C₁₋₆alkyl, C₃₋₆cycloalkyl, -C₁₋₆alkyl-C₃₋₆cycloalkyl, C₆₋₁₀aryl, -C₁₋₆alkyl-C₆₋₁₀aryl, C₁₋₉heteroaryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

4. The compound of any one of claims 1-3, or a pharmaceutically acceptable salt or solvate thereof, wherein R⁴ is selected from C₃₋₆cycloalkyl, -C₁₋₆alkyl-C₃₋₆cycloalkyl, C₆₋₁₀aryl, and C₁₋₆alkyl-C₆₋₁₀aryl, wherein C₃₋₆cycloalkyl, -C₁₋₆alkyl-C₃₋₆cycloalkyl, C₆₋₁₀aryl, and -C₁₋₆alkyl-C₆₋₁₀aryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

5. The compound of any one of claims 1-4, or a pharmaceutically acceptable salt or solvate thereof, wherein R⁴ is C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

6. The compound of any one of claims 1-5, or a pharmaceutically acceptable salt or solvate thereof, wherein R⁴ is unsubstituted C₃₋₆cycloalkyl.

7. The compound of any one of claims 1-4, or a pharmaceutically acceptable salt or solvate thereof, wherein R⁴ is -C₁₋₆alkyl-C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

8. The compound of claim 7, or a pharmaceutically acceptable salt or solvate thereof, wherein R⁴ is unsubstituted -C₁₋₆alkyl-C₃₋₆cycloalkyl.

9. The compound of any one of claims 1-4, or a pharmaceutically acceptable salt or solvate thereof, wherein R⁴ is phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

10. The compound of claim 9, or a pharmaceutically acceptable salt or solvate thereof, wherein R⁴ is unsubstituted phenyl.

11. The compound of any one of claims 1-4, or a pharmaceutically acceptable salt or solvate thereof, wherein R⁴ is -C₁₋₆alkyl-phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

12. The compound of claim 11, or a pharmaceutically acceptable salt or solvate thereof, wherein R⁴ is unsubstituted -C₁₋₆alkyl-phenyl.

13. The compound of any one of claims 1-4, or a pharmaceutically acceptable salt or solvate thereof, wherein R⁴ is 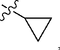 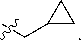 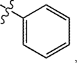 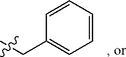 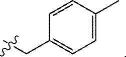
.

14. The compound of any one of claims 1-13, or a pharmaceutically acceptable salt or solvate thereof, wherein R³ is C₁₋₉heteroaryl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

15. The compound of any one of claims 1-14, or a pharmaceutically acceptable salt or solvate thereof, wherein R³ is unsubstituted C₁₋₉heteroaryl.

16. The compound of any one of claims 1-15, or a pharmaceutically acceptable salt or solvate thereof, wherein R³ is 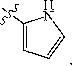 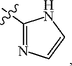 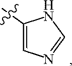 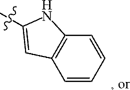 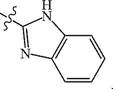
.

17. The compound of any one of claims 1-16, or a pharmaceutically acceptable salt or solvate thereof, wherein R² is selected from C₁₋₆alkyl, -C₁₋₆alkyl-C₃₋₆cycloalkyl, -C₁₋₆alkyl-C₆₋₁₀aryl, and -C₁₋₆alkyl-C₁₋₉heteroaryl, wherein C₁₋₆alkyl, -C₁₋₆alkyl-C₃₋₆cycloalkyl, -C₁₋₆alkyl-C₆₋₁₀aryl, and -C₁₋₆alkyl-C₁₋₉heteroaryl are optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

18. The compound of any one of claims 1-17, or a pharmaceutically acceptable salt or solvate thereof, wherein R² is C₁₋₆alkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

19. The compound of any one of claims 1-18, or a pharmaceutically acceptable salt or solvate thereof, wherein R² is unsubstituted C₁₋₆alkyl.

20. The compound of any one of claims 1-16, or a pharmaceutically acceptable salt or solvate thereof, wherein R² is -C₁₋₆alkyl-C₃₋₆cycloalkyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

21. The compound of claim 20, or a pharmaceutically acceptable salt or solvate thereof, wherein R² is unsubstituted -C₁₋₆alkyl-C₃₋₆cycloalkyl.

22. The compound of any one of claims 1-16, or a pharmaceutically acceptable salt or solvate thereof, wherein R² is -C₁₋₆alkyl-phenyl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

23. The compound of claim 22, or a pharmaceutically acceptable salt or solvate thereof, wherein R² is unsubstituted -C₁₋₆alkyl-phenyl.

24. The compound of any one of claims 1-16, or a pharmaceutically acceptable salt or solvate thereof, wherein R² is -C₁₋₆alkyl-C₁₋₉heteroaryl optionally substituted with one, two, three, or four groups selected from halogen, C₁₋₆alkyl, C₁₋₆haloalkyl, —OR⁵, —N(R⁵)(R⁶), —C(O)R⁷, —C(O)OR⁵, —C(O)N(R⁵)(R⁶), —S(O)₂R⁷, and —S(O)₂N(R⁵)(R⁶)—.

25. The compound of claim 24, or a pharmaceutically acceptable salt or solvate thereof, wherein R² is unsubstituted -C₁₋₆alkyl-C₁₋₉heteroaryl.

26. The compound of any one of claims 1-16, or a pharmaceutically acceptable salt or solvate thereof, wherein R² is —CH₃, —CH₂CH₃, —C(H)(CH₃)₂, —CH₂C(H)(CH₃)₂, 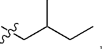 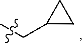 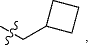 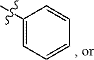 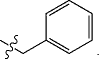
.

27. The compound of any one of claims 1-26, or a pharmaceutically acceptable salt or solvate thereof, wherein R¹ is 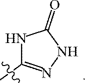
.

28. The compound of any one of claims 1-26, or a pharmaceutically acceptable salt or solvate thereof, wherein R¹ is 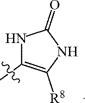
.

29. The compound of any one of claims 1-26, or a pharmaceutically acceptable salt or solvate thereof, wherein R¹ is 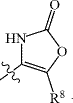
.

30. The compound of any one of claims 1-26, or a pharmaceutically acceptable salt or solvate thereof, wherein R¹ is 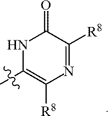
.

31. The compound of any one of claims 1-26, or a pharmaceutically acceptable salt or solvate thereof, wherein R¹ is 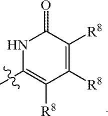
.

32. The compound of any one of claims 1-26, or a pharmaceutically acceptable salt or solvate thereof, wherein R¹ is 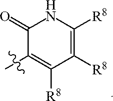
.

33. The compound of any one of claims 1-32, or a pharmaceutically acceptable salt or solvate thereof, wherein each R⁸ is hydrogen.

34. The compound of any one of claims 1-26, or a pharmaceutically acceptable salt or solvate thereof, wherein R¹ is 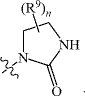
.

35. The compound of any one of claims 1-26, or a pharmaceutically acceptable salt or solvate thereof, wherein R¹ is 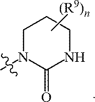
.

36. The compound of any one of claims 1-26, or a pharmaceutically acceptable salt or solvate thereof, wherein R¹ is 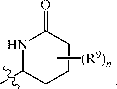
.

37. The compound of any one of claims 34-36, or a pharmaceutically acceptable salt or solvate thereof, wherein n is 0.

38. A compound selected from: 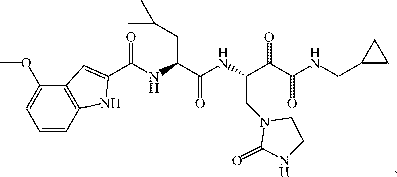 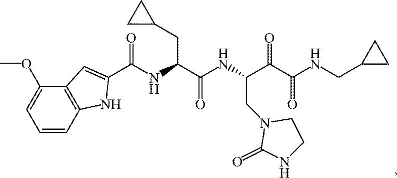 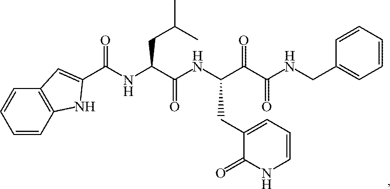 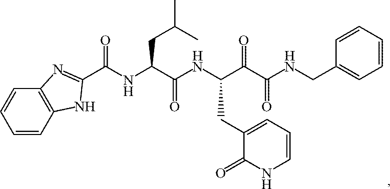 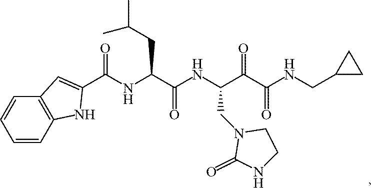 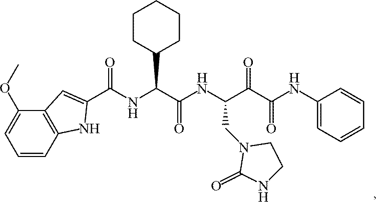 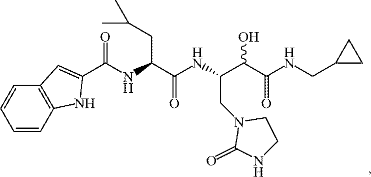 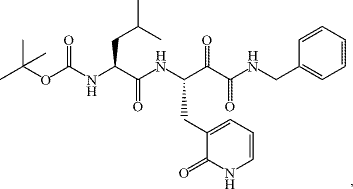 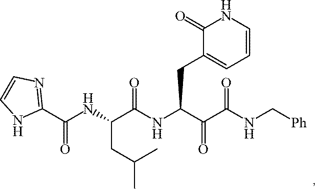 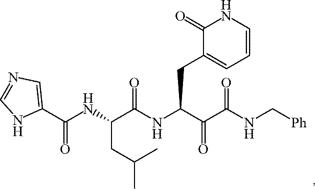 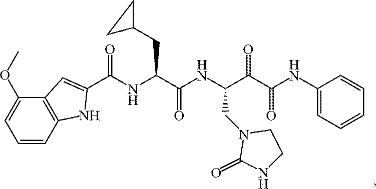 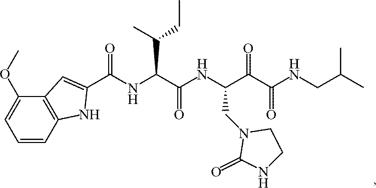 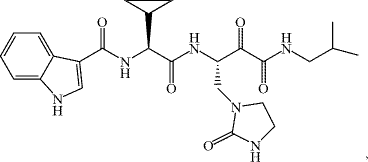 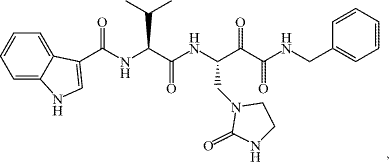 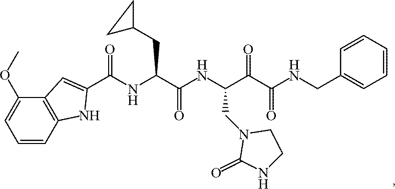 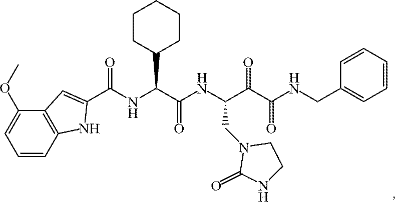 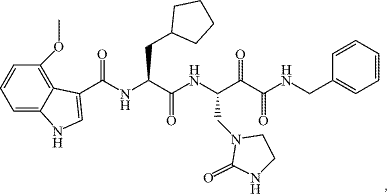 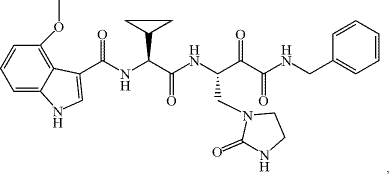 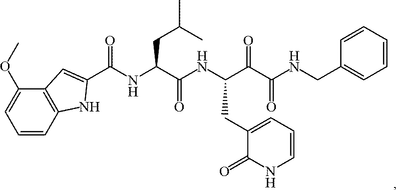 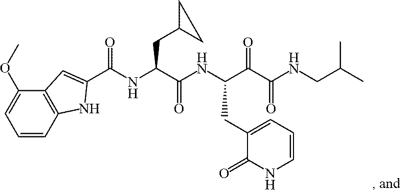 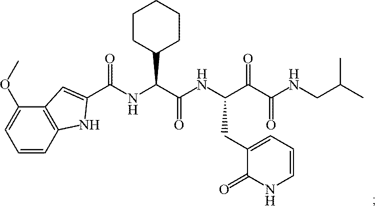
or a pharmaceutically acceptable salt or solvate thereof.

39. A compound selected from: 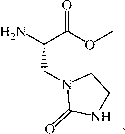 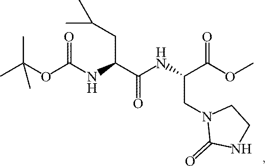 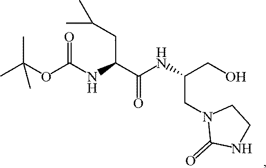 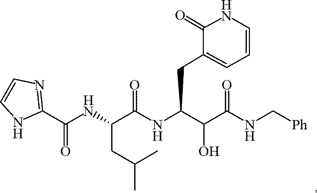 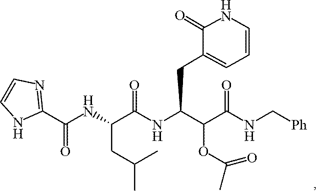 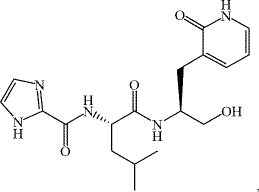 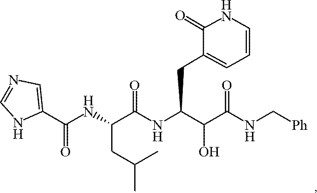 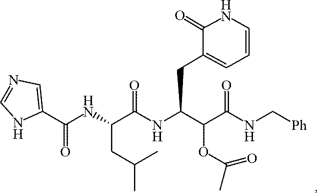 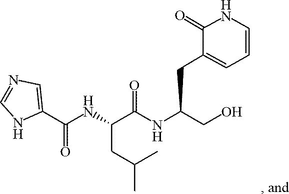 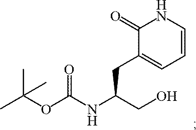
or a pharmaceutically acceptable salt or solvate thereof.

40. A compound selected from: 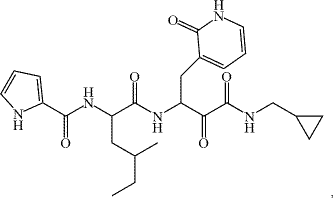 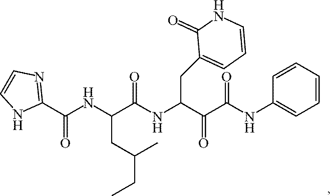 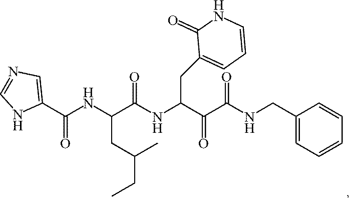 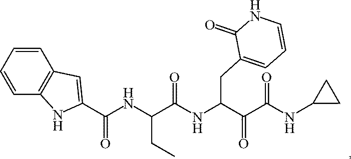 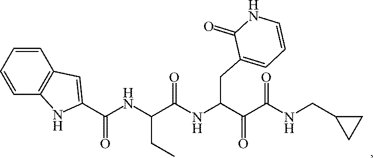 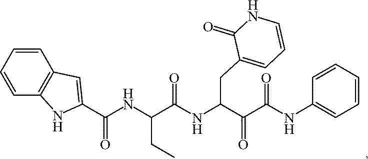 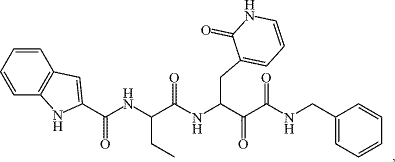 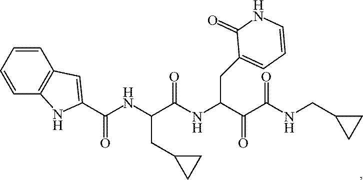 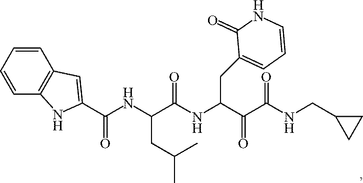 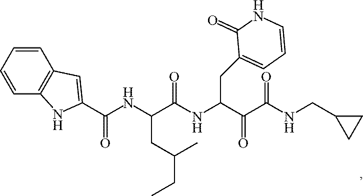 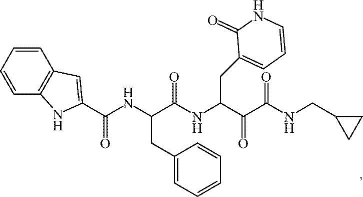 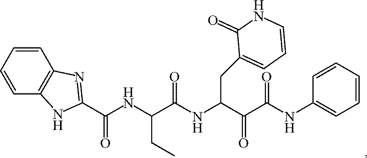 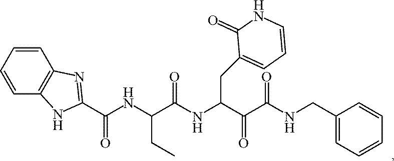 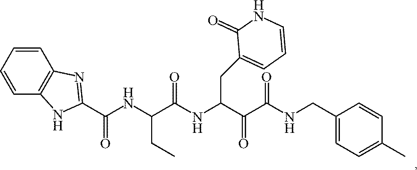 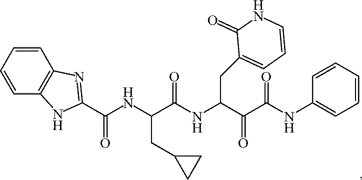 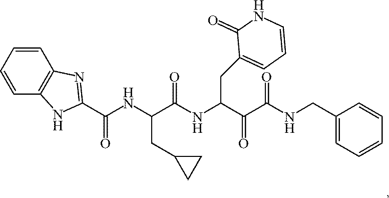 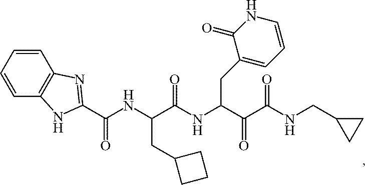 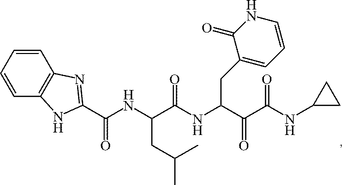 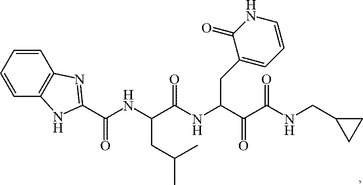 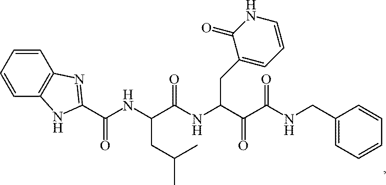 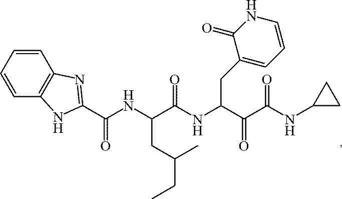 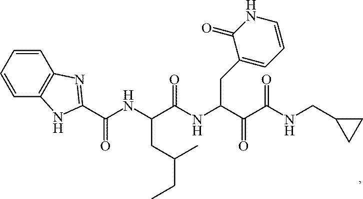 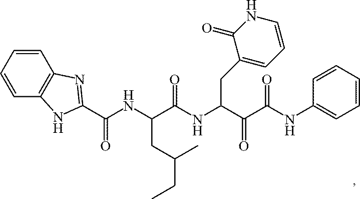 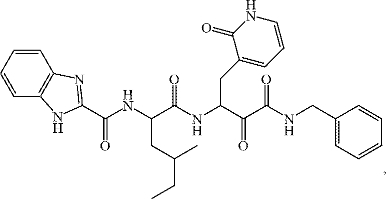 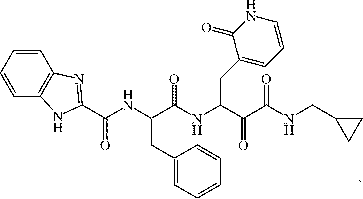 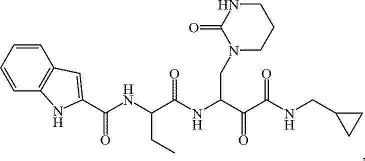 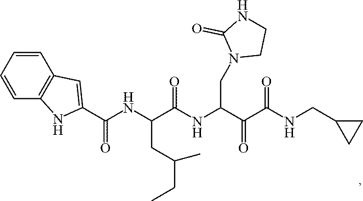 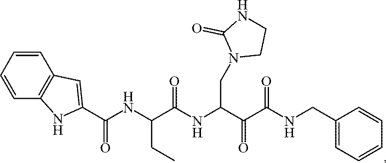 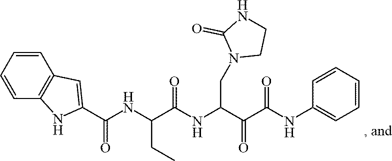 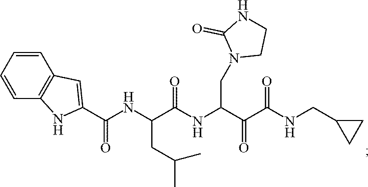
or a pharmaceutically acceptable salt or solvate thereof.

41. A pharmaceutical composition comprising a compound of any one of claims 1-40, or a pharmaceutically acceptable salt or solvate thereof, and at least one pharmaceutically acceptable excipient.

42. A method of treating a coronavirus infection in an individual in need thereof comprising administering to the individual a therapeutically effective amount of a compound of any one of claims 1-40, or a pharmaceutically acceptable salt or solvate thereof.

43. The method of claim 42, wherein the coronavirus infection is severe acute respiratory syndrome coronavirus 2 (SARS-CoV-2).

44. A method of treating COVID-19 in an individual in need thereof comprising administering to the individual a therapeutically effective amount of a compound of any one of claims 1 -40, or a pharmaceutically acceptable salt or solvate thereof.
